(19) United States
(12) Patent Application Publication
(10) Pub. No.: US 2022/0389476 A1
(43) Pub. Date: Dec. 8, 2022

(54) ANIMAL PRODUCT-FREE CULTURE OF STREPTOCOCCUS BACTERIA

(71) Applicant: Vaxcyte, Inc., San Carlos, CA (US)

(72) Inventor: Peter DAVEY, Foster City, CA (US)

(73) Assignee: Vaxcyte, Inc., San Carlos, CA (US)

(21) Appl. No.: 17/747,534

(22) Filed: May 18, 2022

Related U.S. Application Data
(63) Continuation of application No. PCT/US2020/060909, filed on Nov. 17, 2020.
(60) Provisional application No. 62/936,797, filed on Nov. 18, 2019.

(51) Int. Cl. C12Q 1/04; C12N 1/20; C12N 9/08; C12P 19/04

(52) U.S. Cl. CPC C12Q 1/045; C12N 1/205; C12N 9/0065; C12P 19/04; C12R 2001/46

(57) ABSTRACT

The present disclosure provides methods, compositions, and kits for in vitro cultivation of catalase-negative bacteria. The present disclosure further provides catalase-negative bacteria cultivated according to the methods described herein and bacterial stocks thereof.

### CROSS-REFERENCE TO RELATED APPLICATIONS

This application is a Continuation of International Application No. PCT/US2020/060909, filed Nov. 17, 2020, which claims priority to U.S. Provisional Application No. 62/936,797, filed Nov. 18, 2019, the disclosure of each of which is hereby incorporated by reference in its entirety.

### FIELD

This disclosure relates to the fields of microbiology and bacterial culture methods.

### BACKGROUND

Animal-derived materials, such as serum and blood, are frequently used in bacterial cultivation processes. In addition to providing a nutrient-rich environment, the hemoglobin present in animal blood allows aerotolerant or facultative hemolytic bacteria to break down hydrogen peroxide by-products and facilitates bacterial cell growth. However, the use of animal-derived products in bacterial cultivation processes in the context of vaccine production can lead to the introduction of animal derived contaminants, such as prion proteins, mycoplasma, or viruses, into the final bacterial components utilized in vaccine manufacture. Therefore, there is a need in the art for bacterial cultivation methods that do not utilize animal-derived materials.

### SUMMARY

In some embodiments, the present disclosure provides a method of in vitro bacterial cultivation comprising: (a) inoculating an agar medium with catalase-negative bacteria, wherein the agar medium comprises a catalase enzyme and is free of animal-derived materials; and (b) incubating the catalase-negative bacteria on the agar medium under conditions permitting growth of one or more bacterial colonies on the agar medium. In some embodiments, the method further comprises: (c) selecting one of the one or more bacterial colonies from the agar medium; (d) inoculating a liquid medium with the selected bacterial colony to produce a liquid bacterial culture; (e) incubating the liquid bacterial culture under growth-permitting conditions; and (f) harvesting cultivated catalase-negative bacteria from the liquid bacterial culture.

In some embodiments, the catalase-negative bacteria is selected from a Streptococcus spp., a Clostriudium spp., an Aerococcus spp., an Enterococcus spp., a Leuconostoc spp., a Pedioccus spp., an Abiotrophia spp., a Granulicatella spp., a Gemella spp., a Rothia mucilaginosa spp., a Lactococcus spp., a Vagococcus spp., a Helcococcus spp., a Globicatella spp., and a Dolosigranulum spp.

In some embodiments, the catalase-negative bacteria is a Shigella spp. selected from S. dysenteriae Type 1 and S. boydii Type 12.

In some embodiments, the catalase-negative bacteria is selected from Streptococcus spp., Clostriudium spp., Aerococcus spp., and Enterococcus spp. In some embodiments, the Streptococcus spp. is a Group A Streptococcus bacteria, a Group C Streptococcus bacteria, or a viridians Streptococcus bacteria. In some embodiments, the Group A Streptococcus bacteria is S. pyogenes. In some embodiments, the Group A Streptococcus bacteria is of a serotype selected from M1, M3, M4, M12, M28. In some embodiments, the Streptococcus spp. is viridians Streptococcus bacteria selected from the mutans group, the salivarius group, the bovis group, the mitis group, and the anginosus group.

In some embodiments, the Streptococcus spp. is S. pneumonia. In some embodiments, the S. pneumonia is of a serotype selected from the group consisting of 1, 2, 3, 4, 5, 6A, 6B, 7F, 8, 9N, 9V, 10A, 11A, 12F, 14, 15A, 15B, 17F, 18C, 19A, 19F, 20, 22F, 23F, 24F, and 33F. In some embodiments, the S. pneumonia is of a serotype selected from the group consisting of 1, 3, 14, and 19A. In some embodiments, the S. pneumonia is of a serotype selected from the group consisting of 1, 2, 3, 4, 5, 6A, 6B, 6C, 7C, 7F, 8, 9N, 9V, 10A, 11A, 12F, 14, 15A, 15B, 16F, 17F, 18C, 19A, 19F, 20, 20A, 20B, 21, 22F, 23A, 23B, 23F, 24F, 31, 34, 35B, 33F, and 38.

In some embodiments, the Aerococcus spp. is A. viridians.

In some embodiments, the catalase enzyme is present at a concentration of at least about 500 international units (IU). In some embodiments, the catalase enzyme is present at a concentration of about 500 IU to about 10000 IU. In some embodiments, the catalase enzyme is present at a concentration of about 4000 IU to about 6000 IU, about 4500 IU to about 6000 IU, about 5000 IU to about 6000 IU, about 5500 IU to about 6000 IU, about 4000 IU to about 5500 IU, about 4000 IU to about 5000 IU, about 4000 IU to about 4500 IU, about 4500 IU to about 5500 IU, about 4500 IU to about 5000 IU, or about 5000 to about 5500 IU. In some embodiments, the catalase enzyme is present at a concentration of about 4500 IU, about 4600 IU, about 4700 IU, about 4800 IU, about 4900 IU, about 5000 IU, about 5100 IU, about 5200 IU, about 5300 IU, about 5400 IU, or about 5500 IU. In some embodiments, the catalase enzyme is present at a concentration of about 5000 IU.

In some embodiments, the agar medium further comprises a yeast extract, a soy peptone, glucose, one or more salts, and L-cysteine. In some embodiments, the one or more salts are selected from Na₂CO₃, NaCl, and MgSO₄.

In some embodiments, the L-cysteine is present at a concentration of at least about 0.5 g/L. In some embodiments, the L-cysteine is present at a concentration of about 0.5 g/L to about 5 g/L. In some embodiments, the L-cysteine is present at a concentration of about 1 g/L to about 4 g/L. In some embodiments, the L-cysteine is present at a concentration of about 0.5 g/L to about 1.5 g/L. In some embodiments, the L-cysteine is present at a concentration of about 0.5 g/L, 1.0 g/L, 1.5 g/L, 2.0 g/L, 2.5 g/L, 3.0 g/L, 3.5 g/L, 4.0 g/L, 4.5 g/L, or 5.0 g/L.

In some embodiments, the yeast extract is present at a concentration of at least about 5 g/L. In some embodiments, the yeast extract is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L. In some embodiments, the yeast extract is present at a concentration of about 5 g/L, about 10 g/L, about 15 g/L, about 20 g/L, or about 25 g/L.

In some embodiments, the soy peptone is present at a concentration of at least about 5 g/L. In some embodiments, the soy peptone is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L. In some embodiments, the soy peptone is present at a concentration of about 5 g/L, about 10 g/L, about 15 g/L, about 20 g/L, or about 25 g/L.

In some embodiments, the conditions permitting growth of bacterial colonies comprise a temperature of about 37° C. In some embodiments, the conditions permitting growth of bacterial colonies comprise a temperature of between about 34° C. and 39° C. In some embodiments, the conditions permitting growth of bacterial colonies further comprise an anerobic culture environment. In some embodiments, the conditions permitting growth of bacterial colonies further comprise a CO₂ level of at least about 5%. In some embodiments, the CO₂ level is between about 5% and about 95%. In some embodiments, the conditions permitting growth of bacterial colonies further comprise a CO₂ level of about 0%.

In some embodiments, the liquid medium comprises substantially the same components as the agar medium.

In some embodiments, the one or more bacterial colonies comprise opaque, semi-transparent, and transparent colonies. In some embodiments, the selected bacterial colony is an opaque colony.

In some embodiments, the cultivated catalase-negative bacteria is harvested after the liquid bacterial culture reaches a pre-determined optical density (OD) threshold. In some embodiments, the optical density is measured at a wavelength of 600 nm (OD600). In some embodiments, the pre-determined OD threshold is an OD600 of at least about 1.0.

In some embodiments, the present disclosure provides a cultivated catalase-negative bacteria produced by the methods described herein. In some embodiments, the bacteria demonstrate enhanced polysaccharide production compared to a similar bacteria cultivated using media comprising animal-derived materials.

In some embodiments, the present disclosure provides a bacterial stock comprising a cultivated catalase-negative bacteria described herein.

In some embodiments, the present disclosure provides a kit for in vitro bacterial cultivation, comprising: (a) an agar medium that is free of animal-derived materials; and (b) a catalase enzyme. In some embodiments, the kit further comprises a liquid medium comprising substantially the same components as the agar medium.

In some embodiments, the present disclosure provides an agarose plate comprising: (a) an agar medium that is free of animal-derived materials; and (b) a catalase enzyme. In some embodiments, the agarose plate further comprises catalase-negative bacteria.

In some embodiments, the present disclosure provides a bacterial stock comprising cultivated catalase-negative bacteria, a liquid medium, and, optionally, glycerol, wherein the bacterial stock does not comprise an animal-derived material. In some embodiments, the bacterial stock does not comprise animal-derived heme. In some embodiments, the bacterial stock does not comprise a prion protein, mycoplasma, or viruses. In some embodiments, the bacterial stock demonstrates comprises a decreased amount of cell-wall polysaccharide (CWPS) contamination compared to a bacterial stock comprising a similar bacteria cultivated using media comprising animal-derived materials.

### BRIEF DESCRIPTION OF THE DRAWINGS

FIG. 1 shows the OD600 over time of serotype 14 during stage 1 liquid media culture using Media 1, 2, 3, and 4.

FIG. 2. shows the OD600 over time of serotype 1 during stage 1 liquid media culture using Media 1, 2, 3, and 4.

FIG. 3 shows the OD600 over time of serotype 1 during stage 1 and 2 liquid media culture using Media 1 and Media 5.

FIG. 4 shows the OD600 over time of serotype 4 during stage 1 and 2 liquid media culture using Media 1 and Media 5.

FIG. 5 shows the OD600 over time of serotypes 6A and 23F during stage 1 and 2 liquid media culture using Media 5.

FIG. 6 shows the OD600 over time of serotypes 3 and 19A during stage 1 and 2 liquid media culture using Media 5.

FIG. 7 shows the OD600 over time of serotypes 6B, 7F, 9V, and 18C during stage 1 and 2 liquid media culture using Media 5.

FIG. 8 shows the OD600 over time of serotypes 8, 9N, 10A, 11A during stage 1 and 2 liquid media culture using Media 5.

FIG. 9 shows the OD600 over time of serotypes 12F, 15B, 17F, and 19F during stage 1 and 2 liquid media culture using Media 5.

FIG. 10 shows the OD600 over time of serotypes 2, 20, 22F, and 33F during stage 1 and 2 liquid media culture using Media 5.

FIG. 11 shows the OD600 over time of serotypes 15A, 35B, and 23B during stage 1 and 2 liquid media culture using Media 5.

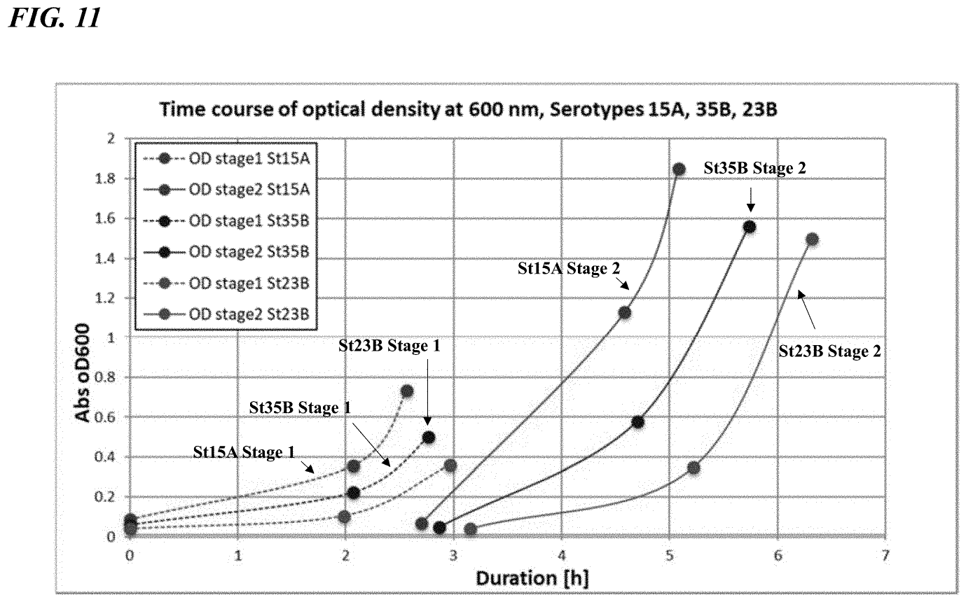

FIG. 12 shows the OD600 over time of serotypes 16F, 7C, and 31 during stage 1 and 2 liquid media culture using Media 5

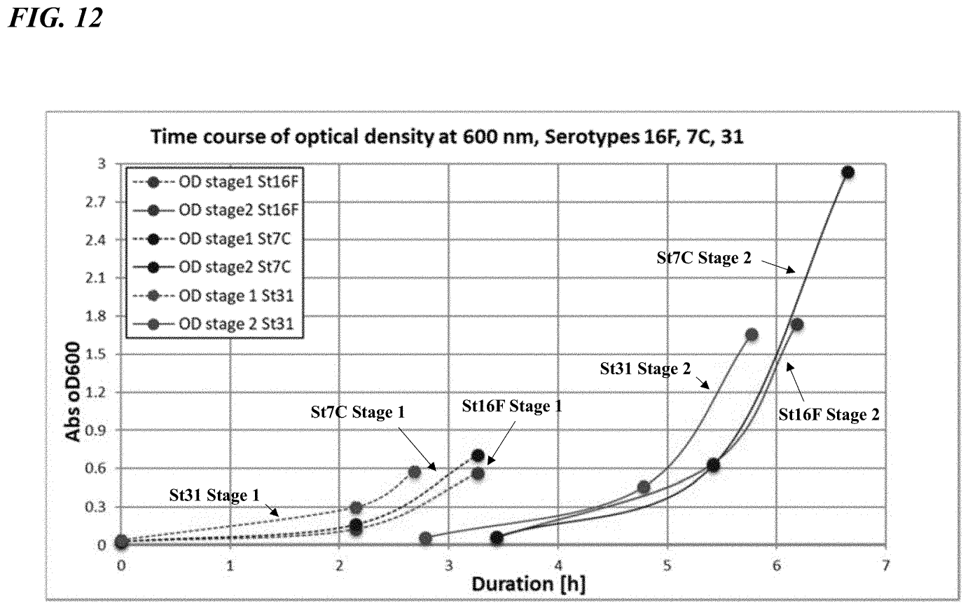

FIG. 13 shows the OD600 over time of serotype 23A during stage 1 and 2 liquid media culture using Media 5.

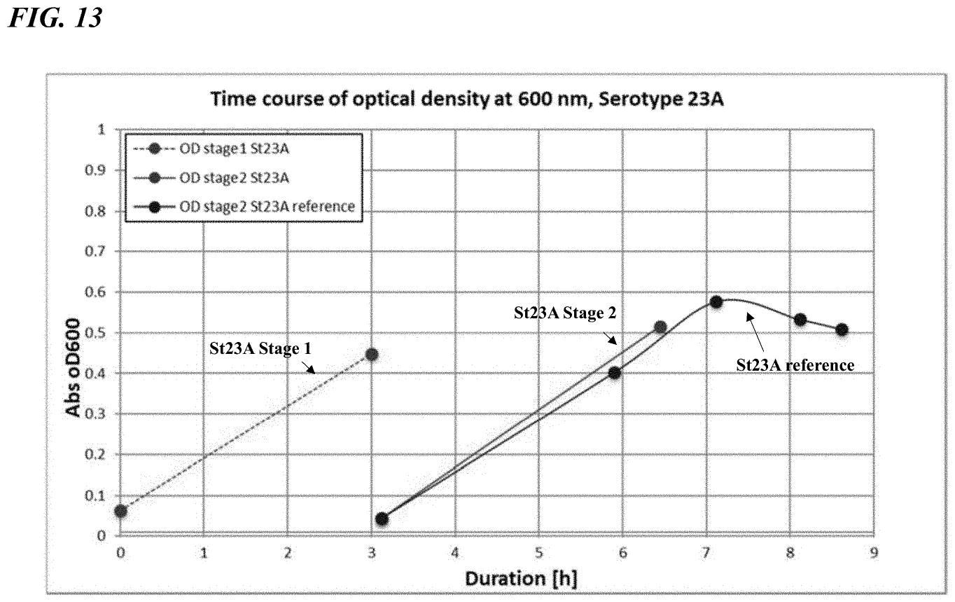

FIG. 14 shows colonies expressing serotype 6A on transparent media.

FIG. 15 shows time courses of OD600 of serotype 20 in heat (HS) and filter (FS) sterilized media during cultivation experiments on 600 mL scale.

### DETAILED DESCRIPTION

### Overview

Bacterial growth in an aerobic environment leads to the formation of reactive oxygen species. Reactive oxygen species (ROS), such as superoxide (O₂—), are damaging to cellular membranes and DNA and therefore inhibit growth. Bacterial cells have evolved to express a superoxide dismutase enzyme to convert superoxide to hydrogen peroxide. Unfortunately, hydrogen peroxide is reactive and causes damage to bacterial cells. Therefore, in order to grow in aerobic environments, there must be a mechanism by which the bacteria can break down hydrogen peroxide into water and oxygen in order to prevent bacterial cell damage.

One such mechanism is the use of heme groups present in hemoglobin, which catalyze the breakdown of hydrogen peroxide to water and oxygen. Blood agar plates can provide a source of hemoglobin. For example, S. pneumoniae are alpha-hemolytic when plated on blood agar, releasing lytic enzymes to partially hydrolyze red blood cells to release hemoglobin. The zone of hemolysis can be seen around the S. pneumoniae colonies plated on sheep's blood agar plates. When the blood cells on blood agar plates are lysed they release hemoglobin and the heme groups catalyze the breakdown of hydrogen peroxide to water and oxygen, thereby allowing S. pneumoniae to grow on plates in an aerobic environment.

Another such mechanism is the use of catalase, an enzyme that breaks down hydrogen peroxide to water and oxygen. The protein structure of catalase contains heme groups that promote this activity. Several catalase-positive bacteria are known, including Staphylococci and Micrococci spp. Other bacteria are catalase-negative, for example Streptococcus and Enterococcus spp. and will not grow in an aerobic environment on general lab media that do not contain hemoglobin. While catalase-negative bacteria can be grown on blood agar plates, this increases the risk of contamination by prion proteins that can lead to chronic neurodegenerative diseases such as Transmissible Spongiform Encephalopathies (TSE) and Bovine Spongiform Encephalopathy (BSE).

The World Health Organization has published guidance for vaccine manufacturers on the use of animal-derived materials, such as blood, in the manufacture of vaccines and encourages manufacturers, whenever possible, to avoid the use of materials of animal origin. (WHO Report 927 on Conjugate vaccines). If materials of animal origin are required, they should be sourced from tissues with low infectivity (IB) or no infectivity (IC) and materials should be sourced from a country with no known infectivity (i.e. New Zealand). The relative infectivity levels of various tissues are provided below in Table 1.

TABLE 1
Infectivity Categories of Tissue Samples

| Category | Tissue or Fluid |
|---|---|
| A - High Infectivity | Nervous System: Brain, skull, vertebral column, spinal and |
| | trigeminal ganglia, retina and optic nerve, pituitary gland, dura |
| | mater |
| | Alimentary Tract: Small intestines (duodenum, jejunum, ileum) |
| | including intestinal mucosa. |
| | Lymphoreticular Tissues: Tonsils |
| B - Lower Infectivity | Alimentary Tract: Esophagus, fore-stomach (ruminants only), |
| | stomach, large intestine |
| | Lymphoreticular Tissues: Spleen, lymph nodes, nicitating |
| | membrane, thymus |
| | Body Fluids: Cerebrospinal fluid, blood |
| | Other Tissues: Lungs, liver, kidney, adrenal, pancreas, bone |
| | marrow, blood vessels, olfactory mucosa, gingival tissue, salivary |
| | gland, cornea |
| C - Tissues with No | Reproductive Tissues: Testes, prostate/epididymis/seminal |
| Detected Infectivity | vesicle, semen, ovary, uterus, placenta fluids, fetus, embryos |
| | Musculo-skeletal Tissues: Bones, skeletal muscle, tongue, heart |
| | (pericardium) tendon |
| | Body fluids: Milk, colostrum, cord blood, saliva, sweat, tears, |
| | nasal mucous, urine, feces |

The methods and compositions provided herein enable the cultivation of catalase-negative bacteria without the use of animal-derived materials that can result in unwanted contamination of final bacterial products used in pharmaceutical and biological products. While previous methods have been described using bovine-derived catalase, the methods provided herein allow cultivation of catalase-negative bacteria using 100% animal free media, thereby reducing BSE/TSE concerns described above. The methods further enable selection of bacterial colonies utilizing phase variation techniques and allow for media comprising the same components to be used during the plating and colony selection phases, as well as the fermentation phases. This reduces the likelihood of failed growth during fermentation due to changes in the media, an element that is not possible with blood agar, hemin, or other animal-derived materials.

Use of the methods described and claimed herein may also enable the selection of bacterial colonies with improved polysaccharide productivity when cultured. Cultures with improved polysaccharide productivity may have the benefits of improved efficiency and/or cost effectiveness in polysaccharide production. For example, improved efficiency may be a result of faster growth of the bacteria in culture prior to harvesting, improved conversion rate between media feedstock and polysaccharide obtained, higher polysaccharide yield per liter of fermentation broth, etc.

### Catalase-Negative Bacteria

In some embodiments, the present disclosure provides methods, compositions, and kits for the in vitro cultivation of catalase-negative bacteria. “Catalase-negative bacteria” refers to bacteria that do not express the enzyme catalase and that are identified as negative by the common catalase test, described below and further described in Reiner et al., “Catalase test protocol”, American Society for Microbiology, ASMMicrobeLibrary (2010).

Catalase is a common enzyme found in a variety of living organisms that catalyzes the decomposition of hydrogen peroxide to water and oxygen, thereby protecting the cell from oxidative damage by reactive oxygen species. Catalase has one of the highest turnover numbers of all enzymes; one catalase molecule can convert millions of hydrogen peroxide molecules to water and oxygen each second. Catalase is a tetramer of four polypeptide chains, each over 500 amino acids long. It contains four iron-containing heme groups that allow the enzyme to react with the hydrogen peroxide. The optimum pH for human catalase is approximately 7, and it has a fairly broad maximum: the rate of reaction does not change appreciably between pH 6.8 and 7.5. The pH optimum for catalases from other species varies between 4 and 11 depending on the species. The optimum temperature also varies by species.

The catalase test is one of the three main tests used by microbiologists to identify species of bacteria. If the bacteria possess catalase (i.e., are catalase-positive), bubbles of oxygen are observed when a small amount of bacterial isolate is added to hydrogen peroxide. The catalase test is done by placing a drop of hydrogen peroxide on a microscope slide. An applicator stick is touched to the colony, and the tip is then smeared onto the hydrogen peroxide drop.

If the mixture produces bubbles or froth, the organism is said to be “catalase-positive”. Staphylococci and Micrococci are catalase-positive. Other catalase-positive organisms include Listeria, Corynebacterium diphtheriae, Burkholderia cepacia, Nocardia, the family Enterobacteriaceae (Citrobacter, E. coli, Enterobacter, Klebsiella, Shigella, Yersinia, Proteus, Salmonella, Serratia), Pseudomonas, Mycobacterium tuberculosis, Aspergillus, Cryptococcus, and Rhodococcus equi.

If the mixture does not produce bubbles or froth, the organism is “catalase-negative”. Streptococcus spp., Clostriudium spp., Aerococcus spp., Enterococcus spp., Leuconostoc spp., Pedioccus spp., Abiotrophia spp., Granulicatella spp., Gemella spp., Rothia mucilaginosa spp., Lactococcus spp., Vagococcus spp., Helcococcus spp., Globicatella spp., and Dolosigranulum spp. are examples of catalase-negative bacteria.

In some embodiments, the present disclosure provides methods, compositions kits for the in vitro cultivation of catalase-negative bacteria. In some embodiments, the catalase negative bacteria is an anaerobic bacteria. The term “anaerobe” refers to an organism that does not require oxygen for growth. The term includes obligate anaerobes, which can react negatively (e.g., die) in the presence of oxygen, as well as facultative anaerobes, which can grow in the absence of oxygen and can make ATP by aerobic respiration if oxygen is present.

In some embodiments, the catalase-negative bacteria is selected from a Streptococcus spp., a Clostriudium spp., an Aerococcus spp., an Enterococcus spp., a Leuconostoc spp., a Pedioccus spp., an Abiotrophia spp., a Granulicatella spp., a Gemella spp., a Rothia mucilaginosa spp., a Lactococcus spp., a Vagococcus spp., a Helcococcus spp., a Globicatella spp., and a Dolosigranulum spp. In some embodiments, the catalase-negative bacteria is a Shigella spp. selected from S. dysenteriae Type 1 and S. boydii Type 12.

In some embodiments, the catalase-negative bacteria is selected from a Streptococcus spp., a Clostriudium spp., an Aerococcus spp., and an Enterococcus spp. In some embodiments, the Aerococcus spp. is A. viridians. In some embodiments, the Streptococcus spp. is a Group A Streptococcus bacteria, a Group C Streptococcus bacteria, or a viridians Streptococcus bacteria.

In some embodiments, the Group A Streptococcus bacteria is S. pyogenes. In some embodiments, the Group A Streptococcus bacteria is of a serotype selected from M1, M3, M4, M12, M28.

In some embodiments, the Streptococcus spp. is viridians Streptococcus bacteria selected from the mutans group, the salivarius group, the bovis group, the mitis group, and the anginosus group. In some embodiments, the Streptococcus spp. is S. pneumonia. In some embodiments, the S. pneumonia is of a serotype selected from the group consisting of 1, 2, 3, 4, 5, 6A, 6B, 7F, 8, 9N, 9V, 10A, 11A, 12F, 14, 15A, 15B, 17F, 18C, 19A, 19F, 20, 22F, 23F, 24F, and 33F. In some embodiments, the S. pneumonia is of a serotype selected from the group consisting of 1, 3, 14, and 19A. In some embodiments, the S. pneumonia is of a serotype selected from the group consisting of 1, 2, 3, 4, 5, 6A, 6B, 6C, 7C, 7F, 8, 9N, 9V, 10A, 11A, 12F, 14, 15A, 15B, 16F, 17F, 18C, 19A, 19F, 20, 20A, 20B, 21, 22F, 23A, 23B, 23F, 24F, 31, 34, 35B, 33F, and 38.

### Culture Media

In some embodiments, the present disclosure provides methods of in vitro bacteria cultivation utilizing culture media that are free of animal-derived products. The terms “animal-derived products” and “animal-derived materials” are used interchangeably herein and refer to a product or material that has been purified from an animal or an animal cell. Animal-derived products include blood, serum, growth factors, cytokines, albumin, etc. The culture media of the present disclosure do not comprise animal-derived materials and are thus “animal component-free media” or “animal-free media” These terms are used interchangeably herein and refer to a culture medium that is devoid of any animal-derived materials. Specifically, such medium does not contain any component which has been purified from animals.

The term “culture medium” refers to a liquid or gel (e.g., agar) designed to support the growth of microorganisms or cells Such a medium may be customized to meet specific requirements of growth of the organism and/or the purpose of its growth. The term is inclusive of “agar medium”, which refers to a solid or semi-solid culture medium such as the agar medium used during the initial plating phases of bacterial cultivation (See e.g., Example 1), and “liquid culture medium” such as the liquid medium used in the later growth and fermentation phases of bacterial cultivation (See e.g., Example 2). The terms “liquid medium” and “liquid culture medium” are also used interchangeably throughout the present disclosure.

In some embodiments, the culture media of the present disclosure include agar media and liquid media.

Current good manufacturing practices (GMP) are stringent on quality and selection of several criteria in medium development for microbial fermentation for the production of biologics, especially vaccines. Under GMP fermentation procedures, quality is built into the entire process to ensure that the requirements of regulatory agencies are met in terms of safety, product identity, quality, and purity (FDA Title 21, Code of Federal Regulations, Parts 210, 211, and 600-680). Ideally, the medium should contain only essential components and should be easily prepared in a reproducible manner. Finally, the medium should support the cultivation of the microorganism in question to high-cell density to improve volumetric productivity and to generate a final culture whose composition and physiological condition is suitable for downstream processing. Media development and cultivation protocol development are therefore a vital part of GMP manufacturing.

Various cell culture media for S. pneumoniae have been documented in the literature and a number of media are available commercially. Streptococcus pneumoniae is a fastidious bacterium, growing best in 5% carbon dioxide and complex medium. Nearly 20% of fresh clinical isolates require fully anaerobic conditions. Typically, most of the media used for the growth of fastidious organisms such as S. pneumoniae contain whole blood, (chocolate blood agar, charcoal medium), blood components such as Hemin (Robertson's cooked meat broth), egg yolk (Dorset Egg Media), or other animal materials. These components make the basal media nutritionally enriched and support the growth of the fastidious bacteria.

However, use of blood components or other animal materials in the culture media may pose a serious health hazard due to the increased risk of contaminants like adventitious viruses, prions, and mycoplasma that may get passed on to the final vaccine substance. Furthermore, the animal-derived components (e.g., blood, serum, etc.) are not chemically defined. As such, there may be lot-to-lot variation of these components, thereby introducing lot-to-lot variation in the composition of the culture media. The presence of these animal-derived components in the culture media may further increases the complexity and cost of purification, as the animal-derived proteins will need to be removed.

Thus, there lies a challenge in developing a medium which is free of animal-derived materials and allows for large scale production of the microorganism in high purity and yields.

In some embodiments, the culture media of the present disclosure comprises one or more of a carbon source, a nitrogen source, and a phosphorus source. In some embodiments, the culture media of the present disclosure comprises a catalase enzyme and one or more of a carbon source, a nitrogen source, and a phosphorus source. In some embodiments, the culture media of the present disclosure further comprises one or more salts. Carbon Source

In some embodiments, the culture media of the present disclosure comprise one or more carbon sources selected from, for example, glucose, fructose, lactose, sucrose, maltodextrins, starch, glycerol, vegetable oils such as soybean oil, hydrocarbons, alcohols such as methanol and ethanol, and organic acids such as acetic acid. In some embodiments, the carbon source is selected from glucose, glycerol, lactose, fructose, sucrose, and soybean oil. The term “glucose” includes glucose syrups, e.g., glucose compositions comprising glucose oligomers. The carbon source may be added to the culture as a solid or liquid. The amounts carbon sources added to the culture media are those such as known by the skilled artisan and/or present in commercially available media (See e.g., HiMedia Labs protocol for Glucose agar, available at HiMedia Labs website, catalog #M1589).

In some embodiments, the carbon source is glucose. In some embodiments, the glucose is present at a concentration of at least about 5 g/L. In some embodiments, the glucose is present at a concentration of between about 5 g/L and about 20 g/L, about 5 g/L and about 15 g/L, about 5 g/L and about 10 g/L, about 10 g/L and about 20 g/L, about 15 g/L and about 20 g/L, or about 10 g/L and about 15 g/L. In some embodiments, the glucose is present at a concentration of about 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, or about 20 g/L.

### Nitrogen Source

In some embodiments, the culture media of the present disclosure comprise one or more nitrogen sources selected from, for example, urea, ammonium hydroxide, ammonium salts (such as ammonium sulphate, ammonium phosphate, ammonium chloride, and ammonium nitrate), other nitrates, amino acids such as glutamate and lysine, yeast extract, yeast autolysates, yeast nitrogen base, protein hydrolysates (including, but not limited to peptones, casein hydrolysates such as tryptone and casamino acids), soybean meal, Hy-Soy, tryptic soy broth, cotton seed meal, malt extract, corn steep liquor, and molasses. The amounts of nitrogen sources added to the culture media are those such as known by the skilled artisan and/or present in commercially available media. (See e.g., HiMedia Labs protocol for Glucose agar, available at HiMedia Labs website, catalog #M456 and Cold Spring Harbor Protocols for LB liquid medium, available at Cold Spring Harb Protoc; 2006; doi:10.1101/pdb.rec8141).

In some embodiments, the nitrogen source is a yeast extract. In some embodiments, the yeast extract is present at a concentration of at least about 5 g/L. In some embodiments, the yeast extract is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L. In some embodiments, the yeast extract is present at a concentration of about 5 g/L, about 10 g/L, about 15 g/L, about 20 g/L, or about 25 g/L.

In some embodiments, the nitrogen source is a soy peptone. In some embodiments, the soy peptone is present at a concentration of at least about 5 g/L. In some embodiments, the soy peptone is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L. In some embodiments, the soy peptone is present at a concentration of about 5 g/L, about 10 g/L, about 15 g/L, about 20 g/L, or about 25 g/L.

In some embodiments, the nitrogen source is an amino acid such as L-cysteine. In some embodiments, L-cysteine is present at a concentration of at least about 0.5 g/L. In some embodiments, L-cysteine is present at a concentration of about 0.5 g/L to about 5.0 g/L. In some embodiments, L-cysteine is present at a concentration of about 0.5 g/L to about 5/0 g/L, about 1.0 g/L to about 5.0 g/L, about 2.0 g/L to about 5.0 g/L, about 3.0 g/L to about 5.0 g/L, about 4.0 g/L to about 5.0 g/L, about 1.0 g/L to about 4.0 g/L, about 1.0 g/L to about 3.0 g/L, about 1.0 g/L to about 2.0 g/L, about 2.0 g/L to about 4.0 g/L, about 3.0 g/L to about 4.0 g/L, or about 2.0 g/L to about 3.0 g/L. In some embodiments, L-cysteine is present at a concentration of about 0.5 g/L, about 1.0 g/L, about 1.5 g/L, about 2.0 g/L, about 2.5 g/L, about 3.0 g/L, about 3.5 g/L, about 4.0 g/L, about 4.5 g/L, or about 5.0 g/L

In some embodiments, increasing the concentration of L-cysteine in the medium may promote better growth in flasks. In some embodiments, adding about 1.0 g/L, about 2.0 g/L, or about 3.0 g/L of L-cysteine directly to the medium prior to inoculation is optimal for growth promotion. In some embodiments, adding about 1.0 g/L, about 2.0 g/L, or about 3.0 g/L of L-cysteine directly to the medium prior to inoculation promotes growth without resulting in unwanted precipitation.

### Phosphorus Source

In some embodiments, the culture media of the present disclosure comprise one or more phosphorus sources. The phosphorus may be in the form of a salt, for example, it may be added as a phosphate (such as ammonium phosphate or potassium phosphate) or polyphosphate. If a polyphosphate is used, it may be in the form of a phosphate glass, such as sodium polyphosphate. Such phosphate glasses are useful as their solubility properties are such that concentrated nutrient media can be prepared with no resulting precipitation upon mixing. The amounts of phosphorus sources added to the culture media are those such as known by the skilled artisan and/or present in commercially available media. (See e.g., HiMedia Labs protocol for Glucose agar, available at HiMedia Labs website, catalog #M520).

### Catalase Enzymes

In some embodiments, the culture media of the present disclosure comprise a catalase enzyme. In some embodiments, the culture media comprising the catalase enzyme is an agar media. Preferably, the catalase enzyme is derived from a non-animal source. For example, in some embodiments, the catalase enzyme is derived from Aspergillus niger (UniProt ID: P55303), Aspergillus fumigatus (UniProt ID: Q92405), or E. coli (UniProt ID: P13029). Catalase enzymes are commercially available, for example from Sigma Aldrich, LS Bio, Merck Millipore, and other commercial sources.

In some embodiments, the catalase enzyme is present at a concentration of at least about 500 international units (IU). In some embodiments, the catalase enzyme is present at a concentration of about 500 IU to about 10000 IU. In some embodiments, the catalase enzyme is present at a concentration of about 4000 IU to about 6000 IU, about 4500 IU to about 6000 IU, about 5000 IU to about 6000 IU, about 5500 IU to about 6000 IU, about 4000 IU to about 5500 IU, about 4000 IU to about 5000 IU, about 4000 IU to about 4500 IU, about 4500 IU to about 5500 IU, about 4500 IU to about 5000 IU, or about 5000 to about 5500 IU. In some embodiments, the catalase enzyme is present at a concentration of about 4500 IU, about 4600 IU, about 4700 IU, about 4800 IU, about 4900 IU, about 5000 IU, about 5100 IU, about 5200 IU, about 5300 IU, about 5400 IU, or about 5500 IU. In some embodiments, the catalase enzyme is present at a concentration of about 5000 IU.

### Exemplary Culture Media

In some embodiments, the present disclosure provides an agar medium comprising a catalase enzyme, a yeast extract, a soy peptone, glucose, one or more salts, and L-cysteine. In some embodiments, the one or more salts are selected from NaCl, Na₂CO₃, and MgSO₄. In some embodiments, the agar medium further comprises a HEPES solution (4-(2-hydroxyethyl)-1-piperazineethanesulfonic acid).

In some embodiments, the agar medium comprises a catalase enzyme present at a concentration of between about 4000 international units (IU) and 6000 IU, a yeast extract present at a concentration of at least about 2.5 g/L to about 7.5 g/L, a soy peptone present at a concentration of between about 5 g/L and about 15 g/L, NaCl present at a concentration of at least about 2.5 g/L to about 7.5 g/L, Na₂CO₃ present at a concentration of at least about 0.05 g/L to about 0.20 g/L, MgSO₄ present at a concentration of at least about 0.25 g/L to about 1.0 g/L, L-cysteine present at a concentration of at least about 0.25 g/L to about 1.0 g/L, and glucose. In some embodiments, the agar medium comprises a catalase enzyme present at a concentration of about 5000 IU, a yeast extract present at a concentration of about 5 g/L, a soy peptone present at a concentration of about 10 g/L, NaCl present at a concentration of about 5 g/L, Na₂CO₃ present at a concentration of about 0.10 g/L, MgSO₄ present at a concentration of about 0.5 g/L, L-cysteine present at a concentration of about 0.5 g/L, and glucose.

In some embodiments, the agar medium comprises a catalase enzyme present at a concentration of between about 4000 international units (IU) and 6000 IU, a yeast extract present at a concentration of at least about 5 g/L to about 15 g/L, a soy peptone present at a concentration of between about 10 g/L and about 30 g/L, Na₂CO₃ present at a concentration of at least about 0.05 g/L to about 0.20 g/L, MgSO₄ present at a concentration of at least about 0.25 g/L to about 1.0 g/L, L-cysteine present at a concentration of at least about 0.25 g/L to about 1.0 g/L, and glucose. In some embodiments, the agar medium comprises a catalase enzyme present at a concentration of about 5000 IU, a yeast extract present at a concentration of about 10 g/L, a soy peptone present at a concentration of about 20 g/L, Na₂CO₃ present at a concentration of about 0.10 g/L, MgSO₄ present at a concentration of about 0.5 g/L, L-cysteine present at a concentration of about 0.5 g/L, and glucose.

In some embodiments, the agar medium comprises a catalase enzyme present at a concentration of between about 4000 international units (IU) and 6000 IU, a yeast extract present at a concentration of at least about 10 g/L to about 30 g/L, a soy peptone present at a concentration of between about 5 g/L and about 15 g/L, Na₂CO₃ present at a concentration of at least about 0.1 g/L to about 1.0 g/L, L-cysteine present at a concentration of at least about 0.5 g/L to about 2.0 g/L, glucose, and a HEPES solution present at a concentration of at least about 40 g/L to about 50 g/L. In some embodiments, the agar medium comprises a catalase enzyme present at a concentration of about 5000 IU, a yeast extract present at a concentration of about 20 g/L, a soy peptone present at a concentration of about 10 g/L, MgSO₄ present at a concentration of about 0.5 g/L, L-cysteine present at a concentration of about 1.0 g/L, glucose, and a HEPES solution present at a concentration of about 47 g/L.

In some embodiments, the present disclosure provides a liquid culture medium comprising a yeast extract, a soy peptone, glucose, one or more salts, L-cysteine, and a HEPES solution (4-(2-hydroxyethyl)-1-piperazineethanesulfonic acid). In some embodiments, the one or more salts are selected from Na₂CO₃, and MgSO₄. In some embodiments, the liquid medium further comprises a potassium phosphate buffer.

In some embodiments, the liquid media comprise a yeast extract present at a concentration of at least about 10 g/L to about 30 g/L, a soy peptone present at a concentration of at least about 5 g/L to about 15 g/L, Na₂CO₃ present at a concentration of at least about 0.1 to about 1.0 g/L, L-cysteine present at a concentration of at least about 0.5 g/L to about 2.0 g/L, a HEPES solution present at a concentration of at least about 40 g/L to about 50 g/L, and glucose.

In some embodiments, the liquid media comprise a yeast extract present at a concentration of about 20 g/L, a soy peptone present at a concentration of about 10 g/L, Na₂CO₃ present at a concentration of about 0.4 g/L, L-cysteine present at a concentration of about 1.0 g/L, a HEPES solution present at a concentration of about 47 g/L, and glucose.

In some embodiments, the liquid media comprise a yeast extract present at a concentration of at least about 10 g/L to about 30 g/L, a soy peptone present at a concentration of at least about 5 g/L to about 15 g/L, Na₂CO₃ present at a concentration of at least about 0.1 to about 1.0 g/L, L-cysteine present at a concentration of at least about 2 g/L to about 8.0 g/L, a HEPES solution present at a concentration of at least about 40 g/L to about 50 g/L, and glucose. In some embodiments, the liquid media comprise a yeast extract present at a concentration of about 20 g/L, a soy peptone present at a concentration of about 10 g/L, Na₂CO₃ present at a concentration of about 0.4 g/L, L-cysteine present at a concentration of about 4.0 g/L, a HEPES solution present at a concentration of about 47 g/L, and glucose.

In some embodiments, the liquid media comprise a yeast extract present at a concentration of at least about 10 g/L to about 30 g/L, a soy peptone present at a concentration of at least about 5 g/L to about 15 g/L, Na₂CO₃ present at a concentration of at least about 0.1 to about 1.0 g/L, L-cysteine present at a concentration of at least about 2 g/L to about 8.0 g/L, a HEPES solution present at a concentration of at least about 40 g/L to about 50 g/L, and glucose. In some embodiments, the liquid media comprise a yeast extract present at a concentration of about 20 g/L, a soy peptone present at a concentration of about 10 g/L, Na₂CO₃ present at a concentration of about 0.4 g/L, L-cysteine present at a concentration of about 3.0 g/L, a HEPES solution present at a concentration of about 47 g/L, and glucose.

In some embodiments, the liquid media comprise a yeast extract present at a concentration of at least about 10 g/L to about 30 g/L, a soy peptone present at a concentration of at least about 5 g/L to about 15 g/L, Na₂CO₃ present at a concentration of at least about 0.1 to about 1.0 g/L, L-cysteine present at a concentration of at least about 0.5 g/L to about 2.0 g/L, a HEPES solution present at a concentration of at least about 40 g/L to about 50 g/L, glucose, and a potassium phosphate buffer present at a concentration of at least about 0.01 M to about 0.075 M. In some embodiments, the liquid media comprise a yeast extract present at a concentration of about 20 g/L, a soy peptone present at a concentration of about 10 g/L, Na₂CO₃ present at a concentration of about 0.4 g/L, L-cysteine present at a concentration of about 1.0 g/L, a HEPES solution present at a concentration of about 47 g/L, glucose, and a potassium phosphate buffer present at a concentration of about 0.05 M.

In some embodiments, the liquid media comprise a yeast extract present at a concentration of at least about 10 g/L to about 30 g/L, a soy peptone present at a concentration of at least about 5 g/L to about 15 g/L, Na₂CO₃ present at a concentration of at least about 0.1 to about 1.0 g/L, L-cysteine present at a concentration of at least about 0.5 g/L to about 2.0 g/L, a HEPES solution present at a concentration of at least about 40 g/L to about 50 g/L, glucose, and a potassium phosphate buffer present at a concentration of at least about 0.05 M to about 0.2 M. In some embodiments, the liquid media comprise a yeast extract present at a concentration of about 20 g/L, a soy peptone present at a concentration of about 10 g/L, Na₂CO₃ present at a concentration of about 0.4 g/L, L-cysteine present at a concentration of about 1.0 g/L, a HEPES solution present at a concentration of about 47 g/L, glucose, and a potassium phosphate buffer present at a concentration of about 0.1 M.

### Culture Protocols

In some embodiments, the present disclosure provides a method of in vitro bacterial cultivation comprising inoculating an agar medium with catalase-negative bacteria, wherein the agar medium comprises a catalase enzyme and is free of animal-derived materials; and incubating the catalase-negative bacteria on the agar medium under conditions permitting growth of one or more bacterial colonies on the agar medium. In some embodiments, the method further comprises selecting one of the one or more bacterial colonies from the agar plate; inoculating a liquid medium with the selected bacterial colony to produce a liquid bacterial culture; incubating the liquid bacterial culture under growth-permitting conditions; and harvesting cultivated catalase-negative bacteria from the liquid bacterial culture.

In some embodiments, the present disclosure provides a method of in vitro bacterial cultivation comprising inoculating an agar medium with catalase-negative bacteria, wherein the agar medium comprises a catalase enzyme and is free of animal-derived materials; incubating the catalase-negative bacteria on the agar medium under conditions permitting growth of one or more bacterial colonies on the agar medium; selecting one of the one or more bacterial colonies from the agar plate; inoculating a liquid medium with the selected bacterial colony to produce a liquid bacterial culture; incubating the liquid bacterial culture under growth-permitting conditions; and harvesting cultivated catalase-negative bacteria from the liquid bacterial culture.

In some embodiments, the conditions permitting growth of bacterial colonies and/or the growth permitting conditions for the liquid medium comprise a temperature, amount of CO₂ present in the culture environment, amount of O₂ present in the culture environment, and/or a rate of agitation or aeration, such as the conditions described herein.

In some embodiments, the conditions permitting growth of bacterial colonies and/or the growth permitting conditions for the liquid medium comprise a temperature of between about 34° C. and about 39° C. Thus, it may be necessary to heat or cool the vessel containing the culture to ensure a constant culture temperature is maintained. The temperature may be used to control the doubling time (td), thus for a given culture process, the temperature may be different at different phases. In some embodiments, the conditions permitting growth of bacterial colonies and/or the growth permitting conditions for the liquid medium comprise a temperature of about 34°, about 35°, about 36°, about 37°, about 38°, or about 39° C. In some embodiments, the conditions permitting growth of bacterial colonies and/or the growth permitting conditions for the liquid medium comprise a temperature of about 37° C.

In some embodiments, the conditions permitting growth of bacterial colonies and/or the growth permitting conditions for the liquid medium comprise an anaerobic culture environment. In some embodiments, the conditions permitting growth of bacterial colonies and/or the growth permitting conditions for the liquid medium comprise a CO₂ level of about 0%. In some embodiments, the conditions permitting growth of bacterial colonies and/or the growth permitting conditions for the liquid medium comprise a CO₂ level of at least about 5%. In some embodiments, the conditions permitting growth of bacterial colonies and/or the growth permitting conditions for the liquid medium comprise a CO₂ level between about 5% and about 95%.

In some embodiments, the liquid medium and the agar medium used according to the methods of the present disclosure comprise substantially the same components. For example, in some embodiments, the liquid medium and the agar medium each comprise a yeast extract, a soy peptone, glucose, one or more salts, and L-cysteine, and do not comprise animal-derived materials.

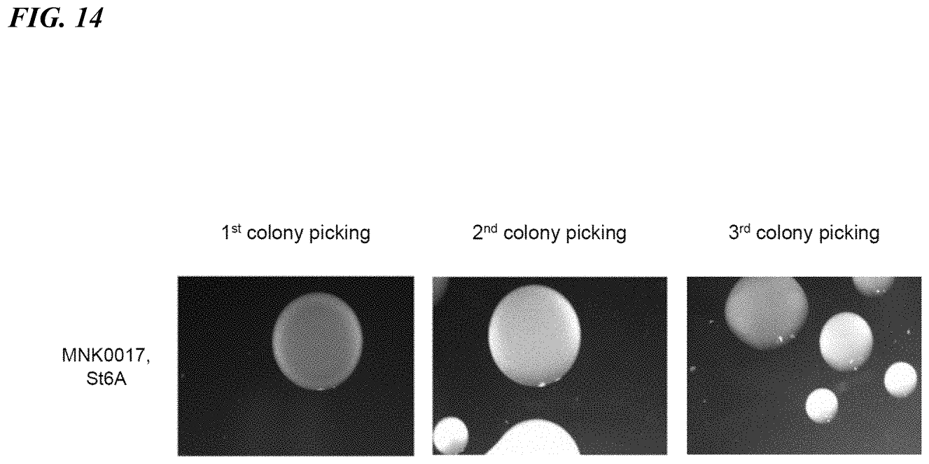

In some embodiments, the one or more bacterial colonies selected from the agar plate are opaque, semi-transparent, or transparent colonies. In some embodiments, the one or more bacterial colonies selected from the agar plate is an opaque colony. In some embodiments, the one or more bacterial colonies are selected using a stereomicroscope. The agar medium of the present disclosure allows for the selection of opaque colonies, which are thought to comprise greater concentrations of the microbial carbohydrates useful in producing glycoprotein conjugate vaccines. The selection of opaque colonies is not possible to perform on traditional blood agar since the blood agar is also opaque. The agar medium of the present disclosure may allow for the selection of colonies with higher concentrations of microbial carbohydrates. FIG. 14 clearly shows opaque colonies growing on the medium of the present disclosure. Selection and use of more productive colonies can lead to greater efficiencies in polysaccharide production.

In some embodiments, the cultivated catalase-negative bacteria is harvested after the liquid bacterial culture reaches a pre-determined optical density (OD) threshold. In some embodiments, the optical density is measured using a spectrophotometer to determine the amount of bacteria present in the liquid culture. In some embodiments, the optical density is measured at a wavelength of 600 nm (OD600). In some embodiments, the pre-determined OD threshold is an OD600 of at least about 1.0.

In some embodiments, the methods described herein utilize multiple rounds of agarose plating and cultivation prior to inoculating the liquid media with a selected bacterial colony. For example, in some embodiments, an agar medium is inoculated with a catalase-negative bacteria and cultured on the agar medium under conditions permitting growth of one or more bacterial colonies. In such embodiments, a bacterial colony is selected from the agar medium and re-suspended in an appropriate buffer solution. A second agar medium is then inoculated with the re-suspended bacteria solution cultured under conditions permitting growth of one or more bacteria colonies on the second agar medium. This process can be repeated a total of 1, 2, 3, 4, 5, or more times to increase the purity of the bacteria used to inoculate the liquid media.

### Compositions and Kits

In some embodiments, the present disclosure provides a cultivated catalase-negative bacteria produced by the methods described herein. The term “cultivated bacteria” refers to a bacterial population that has been produced by in vitro methods. In some embodiments, the cultivated catalase-negative bacteria demonstrate enhanced polysaccharide production compared to similar bacteria cultivated according to other methods. For example, in some embodiments, the cultivated catalase-negative bacteria produced by the methods described herein comprise a polysaccharide content that is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, or about 250% greater than the polysaccharide content of similar bacteria cultivated according to other methods.

In some embodiments, the present disclosure provides a bacterial stock comprising the cultivated catalase-negative bacteria produced by the methods described herein. One or more additional components may be present in the bacterial stock, such as a liquid medium and/or glycerol. In some embodiments, the present disclosure provides a bacterial stock comprising cultivated catalase-negative bacteria, a liquid medium, and, optionally, glycerol, wherein the bacterial stock does not comprise an animal-derived material.

In some embodiments, the bacterial stock does not comprise contaminants such as animal-derived materials. For example, in some embodiments, the bacterial stock does not comprise animal-derived heme, a prion protein, mycoplasma, and/or viruses. In some embodiments, the bacterial stock comprises decreased contaminants such as cell wall polysaccharide (CWPS). For example, in some embodiments, the bacterial stock is substantially free of CWPS contaminants. In some embodiments, the bacterial stock comprises the cultivated catalase-negative bacteria produced by the methods described herein and comprises a decreased amount of CWPS contamination compared to a bacterial stock of a similar bacteria cultivated according to other cultivation methods. In some embodiments, the bacterial stock produced by the methods described herein comprises at least about 20% less CWPS contamination compared to a bacterial stock of a similar bacteria cultivated according to other cultivation methods. In some embodiments, the bacterial stock produced by the methods described herein comprises between about 20% and about 70% less CWPS contamination compared to a bacterial stock of a similar bacteria cultivated according to other cultivation methods. In some embodiments, the bacterial stock produced by the methods described herein comprises about 20%, about 30%, about 40%, about 50%, about 60%, or about 70% less CWPS contamination compared to a bacterial stock of a similar bacteria cultivated according to other cultivation methods.

In some embodiments, the present disclosure provides an agarose plate comprising: an agar medium that is free of animal-derived materials; and a catalase enzyme. In some embodiments, the agarose plate further comprises a catalase-negative bacteria.

In some embodiments, the present disclosure provides kits for carrying out the in vitro bacterial cultivation methods described herein. In some embodiments, a kit can include one or more of the following: one or more culture media (e.g., an agar media and/or a liquid media), one or more agarose plates; a catalase enzyme; one or more reagents for reconstituting and/or diluting the kit components. Components of a kit can be in separate containers or can be combined in a single container. In some embodiments, a kit can include one or more of the following: one or more culture media (e.g., an agar media and/or a liquid media), one or more agarose plates; a catalase enzyme; a bacterial stock; one or more reagents for reconstituting and/or diluting the kit components. Components of a kit can be in separate containers or can be combined in a single container.

In some embodiments, the present disclosure provides a kit for in vitro bacterial cultivation, comprising: an agar medium that is free of animal-derived materials; and a catalase enzyme. In some embodiments, the kit further comprises a liquid medium comprising substantially the same components as the agar medium. In some embodiments, the kit further comprises a bacterial stock of a catalase-negative bacterium.

In addition to above-mentioned components, in some embodiments a kit further comprises instructions for using the components of the kit to practice the methods of the present disclosure. The instructions for practicing the methods are generally recorded on a suitable recording medium. For example, the instructions may be printed on a substrate, such as paper or plastic, etc. As such, the instructions may be present in the kits as a package insert or in the labeling of the container of the kit or components thereof (i.e., associated with the packaging or sub-packaging). In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### FURTHER NUMBERED EMBODIMENTS

Further embodiments of the instant disclosure are provided in the numbered embodiments below:

Embodiment 1. A method of in vitro bacterial cultivation comprising: (a) inoculating an agar medium with catalase-negative bacteria, wherein the agar medium comprises a catalase enzyme and is free of animal-derived materials; and (b) incubating the catalase-negative bacteria on the agar medium under conditions permitting growth of one or more bacterial colonies on the agar medium.

Embodiment 2. The method of Embodiment 1, further comprising: (c) selecting one of the one or more bacterial colonies from the agar medium; (d) inoculating a liquid medium with the selected bacterial colony to produce a liquid bacterial culture; (e) incubating the liquid bacterial culture under growth-permitting conditions; and (f) harvesting cultivated catalase-negative bacteria from the liquid bacterial culture.

Embodiment 3. The method of Embodiment 1 or Embodiment 2, wherein the catalase-negative bacteria is selected from a Streptococcus spp., a Clostriudium spp., an Aerococcus spp., an Enterococcus spp., a Leuconostoc spp., a Pedioccus spp., an Abiotrophia spp., a Granulicatella spp., a Gemella spp., a Rothia mucilaginosa spp., a Lactococcus spp., a Vagococcus spp., a Helcococcus spp., a Globicatella spp., and a Dolosigranulum spp.

Embodiment 4. The method of Embodiment 1 or Embodiment 2, wherein the catalase-negative bacteria is a Shigella spp. selected from S. dysenteriae Type 1 and S. boydii Type 12.

Embodiment 5. The method of Embodiment 1 or Embodiment 2, wherein the catalase-negative bacteria is selected from Streptococcus spp., Clostriudium spp., Aerococcus spp., and Enterococcus spp.

Embodiment 6. The method of Embodiment 3 or Embodiment 5, wherein the Streptococcus spp. is a Group A Streptococcus bacteria, a Group C Streptococcus bacteria, or a viridians Streptococcus bacteria.

Embodiment 7. The method of Embodiment 6, wherein the Group A Streptococcus bacteria is S. pyogenes.

Embodiment 8. The method of Embodiment 6, wherein the Group A Streptococcus bacteria is of a serotype selected from M1, M3, M4, M12, M28.

Embodiment 9. The method of Embodiment 3 or Embodiment 5, wherein the Streptococcus spp. is viridians Streptococcus bacteria selected from the mutans group, the salivarius group, the bovis group, the mitis group, and the anginosus group.

Embodiment 10. The method of Embodiment 3 or Embodiment 5, wherein the Streptococcus spp. is S. pneumonia.

Embodiment 11. The method of Embodiment 10, wherein the S. pneumonia is of a serotype selected from the group consisting of 1, 2, 3, 4, 5, 6A, 6B, 7F, 8, 9N, 9V, 10A, 11A, 12F, 14, 15A, 15B, 17F, 18C, 19A, 19F, 20, 22F, 23F, 24F, and 33F.

Embodiment 12. The method of Embodiment 10, wherein the S. pneumonia is of a serotype selected from the group consisting of 1, 3, 14, and 19A.

Embodiment 13. The method of Embodiment 10, wherein the S. pneumonia is of a serotype selected from the group consisting of 1, 2, 3, 4, 5, 6A, 6B, 6C, 7C, 7F, 8, 9N, 9V, 10A, 11A, 12F, 14, 15A, 15B, 16F, 17F, 18C, 19A, 19F, 20, 20A, 20B, 21, 22F, 23A, 23B, 23F, 24F, 31, 34, 35B, 33F, and 38.

Embodiment 14. The method of Embodiment 3 or Embodiment 5, wherein the Aerococcus spp. is A. viridians.

Embodiment 15. The method of any one of Embodiments 1-14, wherein the catalase enzyme is present at a concentration of at least about 500 international units (IU).

Embodiment 16. The method of any one of Embodiments 1-14, wherein the catalase enzyme is present at a concentration of about 500 IU to about 10000 IU.

Embodiment 17. The method of Embodiment 16, wherein the catalase enzyme is present at a concentration of about 4000 IU to about 6000 IU, about 4500 IU to about 6000 IU, about 5000 IU to about 6000 IU, about 5500 IU to about 6000 IU, about 4000 IU to about 5500 IU, about 4000 IU to about 5000 IU, about 4000 IU to about 4500 IU, about 4500 IU to about 5500 IU, about 4500 IU to about 5000 IU, or about 5000 to about 5500 IU.

Embodiment 18. The method of Embodiment 16, wherein the catalase enzyme is present at a concentration of about 4500 IU, about 4600 IU, about 4700 IU, about 4800 IU, about 4900 IU, about 5000 IU, about 5100 IU, about 5200 IU, about 5300 IU, about 5400 IU, or about 5500 IU.

Embodiment 19. The method of any one of Embodiments 15-18, wherein the catalase enzyme is present at a concentration of about 5000 IU.

Embodiment 20. The method of any one of Embodiments 1-19, wherein the agar medium further comprises a yeast extract, a soy peptone, glucose, one or more salts, and L-cysteine.

Embodiment 21. The method of Embodiment 20, wherein the one or more salts are selected from Na₂CO₃, NaCl, and MgSO₄.

Embodiment 22. The method of Embodiment 20 or Embodiment 21, wherein the L-cysteine is present at a concentration of at least about 0.5 g/L.

Embodiment 23. The method of Embodiment 20 or Embodiment 21, wherein the L-cysteine is present at a concentration of about 0.5 g/L to about 5 g/L.

Embodiment 24. The method of Embodiment 23, wherein the L-cysteine is present at a concentration of about 1 g/L to about 4 g/L.

Embodiment 25. The method of Embodiment 23, wherein the L-cysteine is present at a concentration of about 0.5 g/L to about 1.5 g/L.

Embodiment 26. The method of any one of Embodiments 22-25, wherein the L-cysteine is present at a concentration of about 0.5 g/L, 1.0 g/L, 1.5 g/L, 2.0 g/L, 2.5 g/L, 3.0 g/L, 3.5 g/L, 4.0 g/L, 4.5 g/L, or 5.0 g/L.

Embodiment 27. The method of any one of Embodiments 20-26, wherein the yeast extract is present at a concentration of at least about 5 g/L.

Embodiment 28. The method of Embodiment 27, wherein the yeast extract is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L.

Embodiment 29. The method of Embodiment 27 or Embodiment 28, wherein the yeast extract is present at a concentration of about 5 g/L, about 10 g/L, about 15 g/L, about 20 g/L, or about 25 g/L.

Embodiment 30. The method of any one of Embodiments 20-29, wherein the soy peptone is present at a concentration of at least about 5 g/L.

Embodiment 31. The method of Embodiment 30, wherein the soy peptone is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L.

Embodiment 32. The method of Embodiment 30 or Embodiment 31, wherein the soy peptone is present at a concentration of about 5 g/L, about 10 g/L, about 15 g/L, about 20 g/L, or about 25 g/L.

Embodiment 33. The method of any one of Embodiments 1-32, wherein the conditions permitting growth of bacterial colonies comprise a temperature of about 37° C.

Embodiment 34. The method of any one of Embodiments 1-32, wherein the conditions permitting growth of bacterial colonies comprise a temperature of between about 34° C. and 39° C.

Embodiment 35. The method of any one of Embodiments 1-34, wherein the conditions permitting growth of bacterial colonies further comprise an anaerobic culture environment.

Embodiment 36. The method of any one of Embodiments 1-35, wherein the conditions permitting growth of bacterial colonies further comprise a CO₂ level of at least about 5%.

Embodiment 37. The method of Embodiment 36, wherein the CO₂ level is between about 5% and about 95%.

Embodiment 38. The method of any one of Embodiments 1-35, wherein the conditions permitting growth of bacterial colonies further comprise a CO₂ level of about 0%.

Embodiment 39. The method of any one of Embodiments 2-38, wherein the liquid medium comprises substantially the same components as the agar medium.

Embodiment 40. The method of any one of Embodiments 1-39, wherein the one or more bacterial colonies comprise opaque, semi-transparent, and transparent colonies.

Embodiment 41. The method of any one of Embodiments 2-40, wherein the selected bacterial colony is an opaque colony.

Embodiment 42. The method of any one of Embodiments 2-41, wherein the cultivated catalase-negative bacteria is harvested after the liquid bacterial culture reaches a pre-determined optical density (OD) threshold.

Embodiment 43. The method of Embodiment 42, wherein optical density is measured at a wavelength of 600 nm (OD600).

Embodiment 44. The method of Embodiment 42, wherein the pre-determined OD threshold is an OD600 of at least about 1.0.

Embodiment 45. A cultivated catalase-negative bacteria produced by the method of any one of Embodiments 1-44.

Embodiment 46. The cultivated catalase-negative bacteria of Embodiment 45, wherein the bacteria demonstrate enhanced polysaccharide production compared to a similar bacteria cultivated using media comprising animal-derived materials.

Embodiment 47. A bacterial stock comprising the cultivated catalase-negative bacteria of Embodiment 45 or Embodiment 46.

Embodiment 48. A kit for in vitro bacterial cultivation, comprising: (a) an agar medium that is free of animal-derived materials; and (b) a catalase enzyme.

Embodiment 49. The kit of Embodiment 48, further comprising a liquid medium comprising substantially the same components as the agar medium.

Embodiment 50. An agarose plate comprising: (a) an agar medium that is free of animal-derived materials; and (b) a catalase enzyme.

Embodiment 51. The agarose plate of Embodiment 50, further comprising catalase-negative bacteria.

Embodiment 52. A bacterial stock comprising cultivated catalase-negative bacteria, a liquid medium, and, optionally, glycerol, wherein the bacterial stock does not comprise an animal-derived material.

Embodiment 53. The bacterial stock of Embodiment 52, wherein the bacterial stock does not comprise animal-derived heme.

Embodiment 54. The bacterial stock of Embodiment 52 or Embodiment 53, wherein the bacterial stock does not comprise a prion protein, mycoplasma, or viruses.

Embodiment 55. The bacterial stock of any one of Embodiments 52-54, wherein the bacterial stock demonstrates comprises a decreased amount of cell-wall polysaccharide (CWPS) contamination compared to a bacterial stock comprising a similar bacteria cultivated using media comprising animal-derived materials.

### Examples

### Example 1: Cultivation on Agar Plates

Testing of different growth conditions on different kinds of agar plates were assessed in order to find media and conditions sufficiently supporting growth of S. pneumoniae colonies. Serotypes used in these experiments are listed in Table 2.

TABLE 2
Serotypes used in Plating Experiments

| UAB Name | Confirmed Serotype | SOLOW ID (RCB) |
|---|---|---|
| MNK1173 | 1 | 48/1/02 |
| MNK0240 | 3 | 48/1/03 |
| MNK0330 | 14 | 48/1/15 |
| MNK0359 | 19A | 49/2/03 |

Three solid agar media (YEPD2, PYE2, and SYG) were prepared according to Tables 3, 4, and 5 below.

TABLE 3
Preparation of YEPD2 Agar

| Raw Material | Amount/Concentration | |
|---|---|---|
| Yeast extract | 10 | g/L |
| Soy peptone | 20 | g/L |
| Glucose monohydrate (pre-cooling) | 11 | g/L |
| Na₂CO₃ | 0.11 | g/L |
| MgSO₄ × 7H₂O | 0.5 | g/L |
| L-Cysteine-HCl × H₂O | 0.5 | g/L |
| Purified water | 800 | mL |

Stir until dissolved and add:

| | | |
|---|---|---|
| Bacteriological Agar | 20 | g/L |

| | |
|---|---|
| Purified water | QS to 1000 mL |
| pH adjustment (with 10N NaOH or | 7.5 +/−0.2 |
| 3.7% HCl) | |

Sterilization for 30 min at 122° C. with reference water of similar amount

TABLE 4
Preparation of PYE2 Agar

| Raw Material | Amount/Concentration | |
|---|---|---|
| Yeast extract | 5 | g/L |
| Soy peptone | 10 | g/L |
| NaCl | 5 | g/L |
| Na₂CO₃ | 0.11 | g/L |
| MgSO₄ × 7H₂O | 0.5 | g/L |
| L-Cysteine-HCl × H₂O | 0.5 | g/L |
| Purified water | 800 | mL |

Stir until dissolved and add:

| | | |
|---|---|---|
| Bacteriological Agar | 20 | g/L |

| | |
|---|---|
| Purified water | QS to 1000 mL |
| pH adjustment (with 10N NaOH or | 7.5 +/−0.2 |
| 3.7% HCl) | |

Sterilization for 30 min at 122° C. with reference water of similar amount

TABLE 5
Preparation of SYG Agar

| Raw Material | Amount/Concentration | |
|---|---|---|
| Yeast extract | 20 | g/L |
| Soy peptone | 10 | g/L |
| Glucose 400 g/L (post-cooling) | 25 | mL/L |
| Salt solution | 20 | mL/L |
| Na₂CO₃ | 0.4 | g/L |
| L-Cysteine-HCl × H₂O | 1 | g/L |
| HEPES | 47.7 | g/L |
| Purified water | 700 | mL |

Stir until dissolved and add:

| | | |
|---|---|---|
| Bacteriological Agar | 15 | g/L |

| | |
|---|---|
| Purified water | QS to 975 mL |
| pH adjustment (with 10N NaOH or | 7.5 +/−0.2 |
| 3.7% HCl) | |

Stir and sterilization for 30 min at 122° C. with reference water of similar amount
Cooling to 50° C. (20-40 min) and add:

| | | |
|---|---|---|
| Glucose 400 g/L | 25 | mL/L |

Store in the dark at 2-8° C.

TABLE 6
Salt Solution Preparation

| Raw Material | Amount/Concentration | |
|---|---|---|
| Purified water | 300 | mL |
| CaCl₂ × 2 H₂O | 0.26 | g/L |
| MgSO₄ × 7 H₂O | 0.48 | g/L |

Dissolve both completely and add:

| | | |
|---|---|---|
| Purified water | 500 | mL |
| K₂HPO₄ | 1.0 | g/L |
| KH₂PO₄ | 1.0 | g/L |
| NaHCO₃ | 10 | g/L |
| NaCl | 2.0 | g/L |

| | |
|---|---|
| Purified water | QS to 1000 mL |

Dissolve completely
Filter through 0.22 μm filter

In addition to these plates, different ready-to-use plates or ready-to-use agar mixtures were used as controls or as alternatives to the YEPD2, PYE2, and SYG agars described above. These additional plate and agars are as follows:
(a) TSB (Merck, 1.00550.0500);
(b) TSAII agar with 5% sheep blood (bioMerieux, 43009); and
(c) TSA non-animal free (Merck, 1460150020).

Prior to cell inoculation, catalase (5000 U/plate) was spread on all animal-free agar plates. Catalase was additionally spread on TSA and Blood agar plates as a control to exclude possible negative influence of this solution on cell growth.

Plates were inoculated with bacterial cells in one of four ways:
(a) Cells were scrapped from the surface of the storage vial and directly streaked onto the agar plates;
(b) Cell suspensions were thawed and directly streaked onto the plates;
(c) Cell suspensions were thawed and diluted to 1:5 or 1:10 or 1:100 in 0.9% w/v NaCl solution before streaking onto the plates; or
(d) Cell suspensions were thawed and diluted 1:10 or 1:20 in catalase stock solution prior to streaking onto the plates.

After inoculation, plates were incubated for 16 to 24 hours at 37° C. in either 5% CO₂ or anaerobic conditions.

After incubation, a maximum of 8 colonies were selected for picking. A stereomicroscope was used to identify and select rough/opaque colonies. The picked colonies were resuspended in 1 mL or 2 mL sterile 0.9% w/v NaCl solution.

The bacterial suspensions from the plates were inoculated in different volumes (10 μL, 100 μL, 200 μL) onto a second round of agar plates as follows:
(a) Bacterial suspensions selected from YEPD agar plates were inoculated onto YEPD agar plates and onto positive control plates;
(b) Bacterial suspensions selected from PYE agar plates were inoculated onto PYE agar plates and onto positive control plates;
(c) Bacterial suspensions selected from SYG agar plates were inoculated onto SYG agar plates and onto positive control plates;
(d) Bacterial suspensions selected from positive control plates were inoculated onto PYE, YEPD or SYG plates.

The complete purification process included 4 stages on the agar plates before starting the liquid cultivation.

A summary of the plating, inoculation, environmental conditions, and bacterial growth is provided below in Table 7.

TABLE 7
Summary of Experimental Growth Conditions

| | | Inoculation | | | |
|---|---|---|---|---|---|
| Tested | | Technique and | | Environmental | |
| Serotypes | Plate | Volume | Additives | condition | Growth |
| 19A3 | TSAII agar | Dilution 1:100, 1:10, | +Cat50/−Cat50 | Anaerobic and | Yes |
| | w/5% sheep | 1:5 resuspended cells | | CO₂ | |
| | blood | in NaCl solution | | | |
| | | Direct streaking (of | | | |
| | | scratched cells or | | | |
| | | 10 μL directly from | | | |
| | | thawed vial) | | | |
| | | Dilution 1:10 with | | | |
| | | catalase solution | | | |
| 114 | TSAII agar | 10 μL directly from | +Cat50 | Anaerobic and | Yes |
| | w/5% sheep | thawed vial | | CO₂ | |
| | blood | Dilution 1:5 in 0.9% | | | |
| | TSA not | w/v NaCl | | | |
| | animal free, | Picked 1 or 8 colony (- | | | |
| | Merck | ies), dispersed in 1 | | | |
| | | mL/2 mL 0.9% w/v | | | |
| | | NaCl | | | |
| | | 100 μL spread on the | | | |
| | | next plate | | | |
| 114 | Animal free | 10 μL directly from | +Cat50 | Anaerobic | No |
| | TSB | thawed vial | | | |
| | | Dilution 1:5 in 0.9% | | | |
| | | w/v NaCl | | | |
| | | Picked colony from | | | |
| | | TSA plate, dispersed | | | |
| | | in 1 mL/2 mL 0.9% | | | |
| | | w/v NaCl | | | |
| | | 100 μL spread on the | | | |
| | | next plate | | | |
| 3 | Animal free | Direct streaking | +Cat50 | Anaerobic | Yes |
| | TSB | 10 μL directly from | | | |
| | (medium | thawed vial | | | |
| | does not | Dilution 1:10 with | | | |
| | support | catalase solution | | | |
| | growth of all | | | | |
| | serotypes) | | | | |
| 3 | Animal free | Direct streaking | +Cat50 + | Anaerobic | No |
| | TSB | | 0.05M | | |
| | | | PO₄ | | |
| 19A3 | PYE2 and | Dilution 1:100, 1:10, | +Cat50 | Anaerobic | No |
| | PYE w/yeast | resuspended cells in | | | |
| | | NaCl solution | | | |
| | extract UF of | Direct streaking (of | | | |
| | UF quality | scratched cells or | | | |
| | | 100 μL or 100 μL | | | |
| | | directly from | | | |
| | | thawed vial) | | | |
| 3 | PYE w/yeast | Dilution 1:10 directly | N/A | Anaerobic | No |
| | extract UF of | mixed with Cat50 | | | |
| | UF quality | | | | |
| 19A3 | YEPD2 and | Dilution 1:100, 1:10, | +Cat50 | Anaerobic | No |
| | YEPD w/ | resuspended cells in | | | |
| | yeast extract | NaCl solution | | | |
| | of UF of UF | 100 μL directly from | | | |
| | quality | thawed vial | | | |
| 114 | SYG | 10 μL directly from | +Cat50 | Anaerobic and | Yes |
| | | thawed vial | | CO₂ | |
| 114 | SYG | Dilution 1:10 directly | N/A | Anaerobic and | Yes |
| | | mixed with Cat50 | | CO₂ | |
| 114 | SYG | Picked 1 or 8 | +Cat50 | Anaerobic and | Yes |
| | | colony(ies) from TSA | | CO₂ | |
| | | plate, dispersed in 1 | | | |
| | | mL/2 mL 0.9% w/v | | | |
| | | NaCl, | | | |
| | | 100 μL spread on the | | | |
| | | next plate (2x) | | | |
| 114 | SYG | Picked 1 or 8 | +Cat50 | Anaerobic and | Yes |
| | | colony(s) from SYG | | CO₂ | |
| | | plate, dispersed in 1 | | | |
| | | mL/2 mL 0.9% w/v | | | |
| | | NaCl, | | | |
| | | 100 μL spread on the | | | |
| | | next plate (2x) | | | |

Based on the experiments described above, the following conclusions were made:
(a) YEPD and PYE agars were not suitable for growth of the chosen S. pneumoniae serotypes.
(b) Agar prepared from TSB ready-to-use animal free media supported the growth of the S. pneumoniae serotype 3, but did not support the growth of other serotypes 1 and 14. Therefore, this media was not used further.
(c) SYG agar with catalase supported the growth of all tested serotypes under diverse conditions. Therefore, this agar was chosen for the purification procedure of the 24 different S. pneumoniae serotypes and for the generation of the parent cell bank for each serotype.
(d) Both the TSAII agar with 5% sheep blood and the TSA agar with catalase supported growth of all tested serotypes under all conditions. TSAII agar with 5% sheep blood without addition of the catalase was used as a positive control during the parent cell bank generation, as the S. pneumoniae showed very typical colony growth and had the a hemolysis circle around the colonies on the blood agar plates.
(e) Interestingly, when cultivated on the SYG agar, all tested serotypes showed better growth in an anaerobic chamber than in a CO₂ incubator. Colonies grown on TSAII agar with 5% sheep blood showed better colony growth in a CO₂ incubator than in the anaerobic chamber.

The final procedure for the purification process used the SYG agar plates treated with catalase (5000 U/plate) and the TSAII agar with 5% sheep blood as a positive control. Vials of bacterial stocks were thawed and 10 μL of the cell suspension was diluted in 2 mL NaCl 0.9%.

100 μL of the cell suspension was spread on the TSAII agar with 5% sheep blood positive control plates and plates were cultivated anaerobically and under 5% CO₂ for about 24 hours at 37° C. 100 μL of the cell suspension, as well as dilutions up to 10⁻³, were spread on the SYG agar plates and plates were cultivated anaerobically for about 24 hours at 37° C.

The plating and cultivation procedure was completed three additional times, for a total of four repetitions. From each plate, one single opaque colony (determined by microscopic observation) was resuspended in 2 mL NaCl 0.9% solution. 100 μL of the cell suspension was spread on the TSAII agar with 5% sheep blood positive control plate and cultivated anaerobically and under 5% CO₂ for about 24 hours at 37° C. 100 μL of the cell suspension was diluted between 10⁻² and up to 10⁻⁵ (depending on the colony size) and spread on the SYG agar plate and cultivated anaerobically for about 24 hours at 37° C. At the end of the fourth stage, colonies were scraped from the plates and used as the inoculum for the first liquid culture stage.

### Example 2: Liquid Cultivation of Bacteria selected from Animal-Free Plates

The following conditions were kept the same for all preliminary experiments in liquid media:
(a) Temperature: 37.0±2.0° C.
(b) Atmosphere enriched with CO₂ (5%)
(c) Shaking speed 200 rpm at 2 cm shaking diameter
(d) Starting pH: 7.5±0.2 (pH was adapted during the first experiments with 20% Na₂CO₃ solution)
(e) All measurements of optical density were performed at OD600 with non-inoculated media as a blank.
(f) Tested serotypes: 1, 4 and 14
(g) Colonies from agar plate cultivation stages 2 or 3 were used.

Five liquid media were used in preliminary experiments. Medium 1: SYG liquid medium with 1.0 g/L L-Cysteine (heat sterilized); Medium 2: SYG liquid medium with 1.0 g/L L-Cysteine (filter sterilized); Medium 3: SYG liquid medium with 1.0 g/L L-Cysteine and 0.05 M Phosphate buffer (filter sterilized); Medium 4: SYG liquid medium with 1.0 g/L L-Cysteine and 0.1 M Phosphate buffer (filter sterilized); Medium 5: SYG liquid medium with 4.0 g/L L-Cysteine (filter sterilized). Details for each media are shown in Table 8.

TABLE 8
Liquid Media Components

| | Media | | | | | | | | | |
|---|---|---|---|---|---|---|---|---|---|---|
| Raw material | 1 | | 2 | | 3 | | 4 | | 5 | |
| Purified water | 700 | mL | 700 | mL | 700 | mL | 700 | mL | 700 | mL |
| Yeast extract | 20.0 | g/L | 20.0 | g/L | 20.0 | g/L | 20.0 | g/L | 20.0 | g/L |
| Soy peptone | 10.0 | g/L | 10.0 | g/L | 10.0 | g/L | 10.0 | g/L | 10.0 | g/L |
| Salt solution | 20.0 | mL/L | 20.0 | mL/L | 20.0 | mL/L | 20.0 | mL/L | 20.0 | mL/L |
| NaHCO₃ | 0.4 | g/L | 0.4 | g/L | 0.4 | g/L | 0.4 | g/L | 0.4 | g/L |
| L-cysteine - HCl x H₂O | 1.0 | g/L | 1.0 | g/L | 1.0 | g/L | 1.0 | g/L | 4.0 | g/L |

| | | | | | | | | |
|---|---|---|---|---|---|---|---|---|
| HEPES | 47.7 | g/L | 47.7 | g/L | — | — | 47.7 | g/L |

| | | | | | | | | | |
|---|---|---|---|---|---|---|---|---|---|
| Glucose Monohydrate | — | 10.0 | g/L | 10.0 | g/L | 10.0 | g/L | 10.0 | g/L |

| | | | | | |
|---|---|---|---|---|---|
| Potassium phosphate | — | — | 0.05M | 0.1M | — |
| buffer | | | | | |

Stir until dissolved and add:

| | | | | | |
|---|---|---|---|---|---|
| pH adjustment (w/10N | 7.5 ± 0.3 | 7.5 ±0.3 | 7.5 ± 0.3 | 7.5 ± 0.3 | 7.5 ± 0.3 |
| NaOH) | | | | | |
| Purified water | QS to 975 mL | QS to 1000 mL | QS to 1000 mL | QS to 1000 mL | QS to 1000 mL |

After the pH adjustment, Media 1 was stirred and heat sterilized for >30 min at >122° C., with reference water of similar amount, after which 25 mL/L of 400 g/L glucose was added. After pH adjustment, Media 2-5 were stirred and filtered using a 0.22 μm filter.

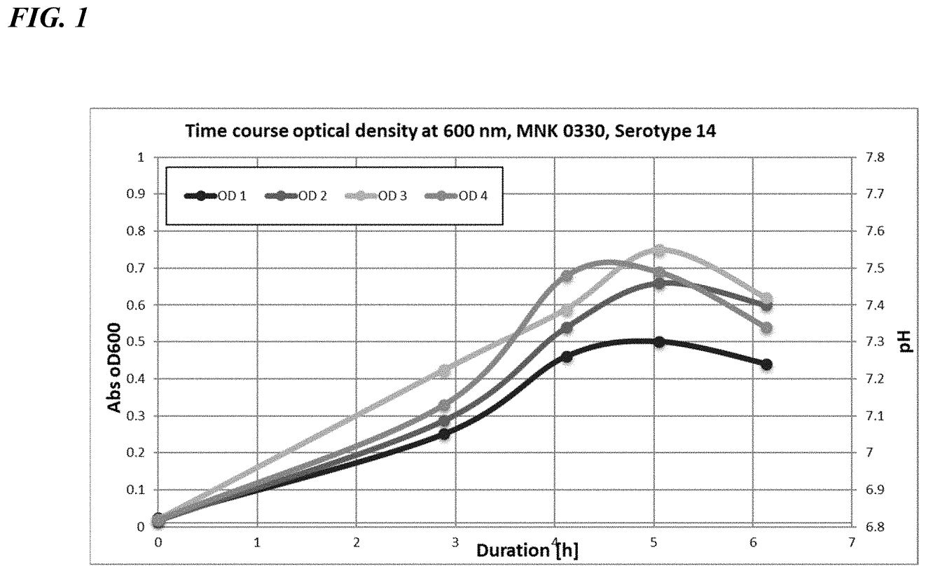

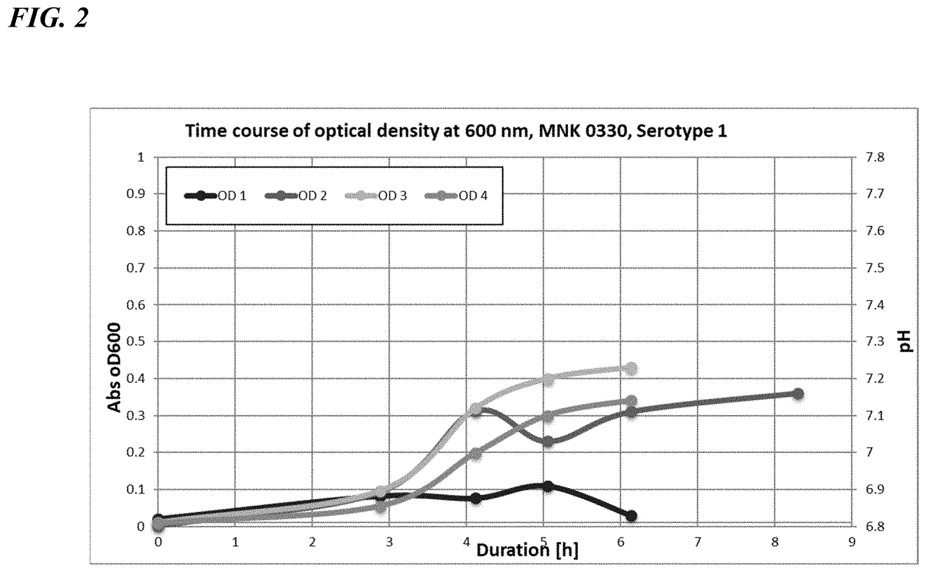

The first experiment was performed with media 1 to 4 and with serotypes 1 and 14 (FIG. 1 and FIG. 2). Colonies from plates were re-suspended in 5 mL 0.9% NaCl solution and 1 mL of the cell suspension was added to 25 mL of the liquid media in a 100 mL shake flask without baffles. The starting OD600 of the liquid culture was between 0.01 and 0.02. Maximal optical density for the first stage was between 0.4 and 0.7. The pH was adjusted once in all shake flasks. The pH adjustment during the first stage of liquid cultivation had an adverse effect on the cell growth. 2 mL of the liquid culture from the first stage was inoculated into a second flask of liquid media for the second liquid culture stage. The growth in the second stage was very slow and the experiment was stopped. Based on this initial liquid culture experiment, it was concluded that the first liquid culture stage should be inoculated with a greater amount of the bacterial cell suspension, no pH adjustment should be done during cultivation, and the growth in the first stage should be in exponential phase prior culture transfer to the second stage.

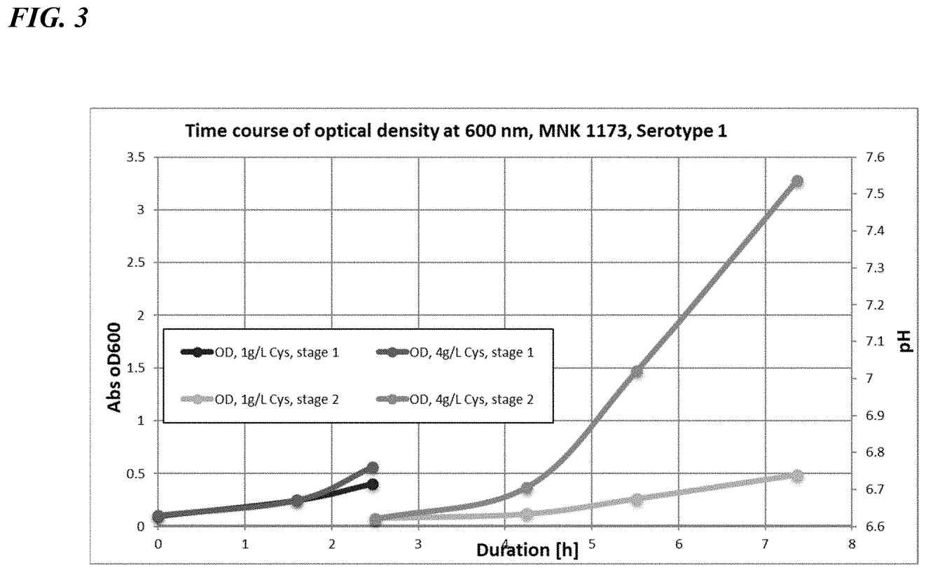

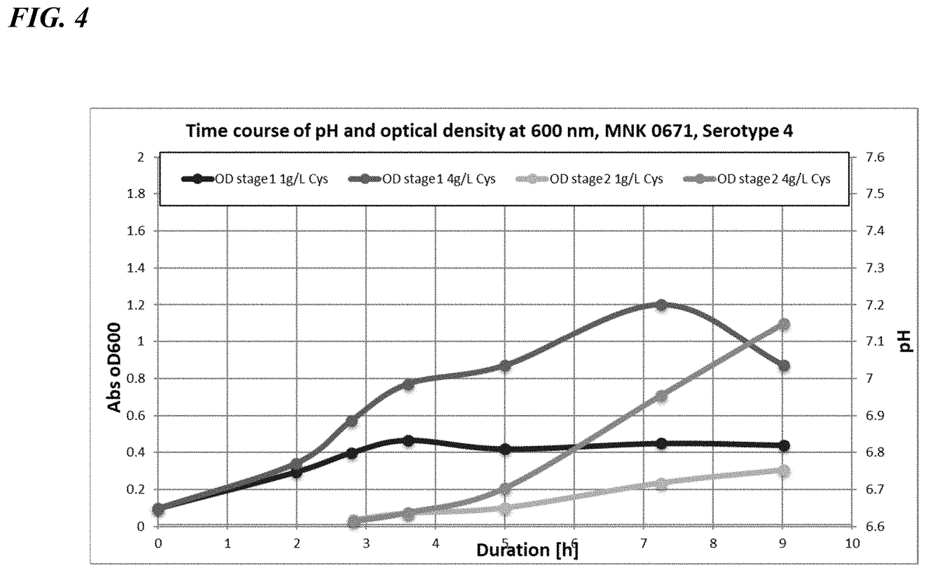

The second group of experiments was performed with media 1 and 5 and with serotypes 1 and 4 (FIG. 3 and FIG. 4). Colonies from plates were re-suspended in 5 mL 0.9% NaCl solution and 4 mL of the cell suspension was added to 100 mL of the media in a 500 mL shake flask without baffles. Starting OD600 of the liquid culture was around 0.1. Maximal optical density for the first liquid culture stage was around 0.4 for media 1 and above 0.5 for media 5. In the second stage, 10 mL of the culture from the first stage was inoculated to 100 mL of the media in a 500 mL shake flask without baffles. Maximal optical density for the second stage of liquid culture was around 0.4 to 0.5 for media 1 and above 1.0 for media 5.

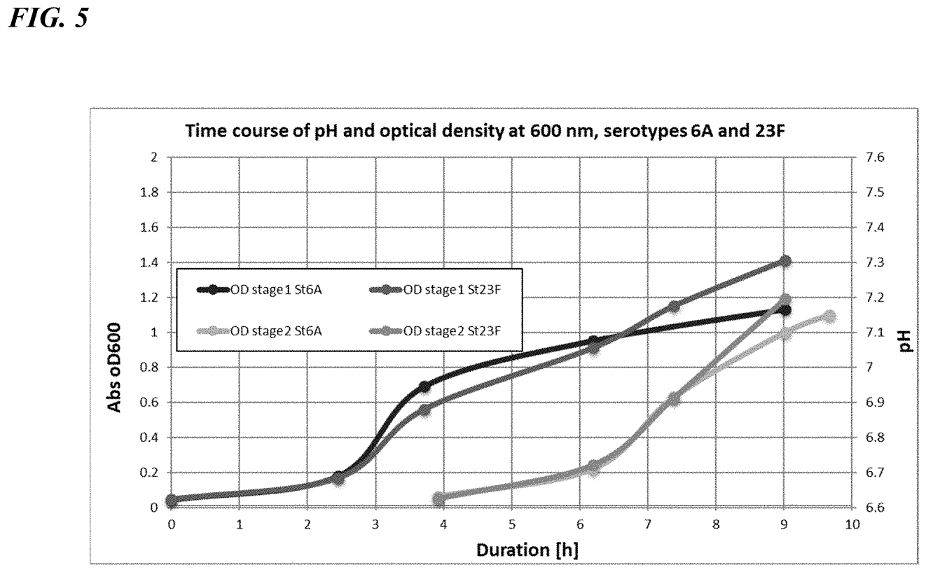

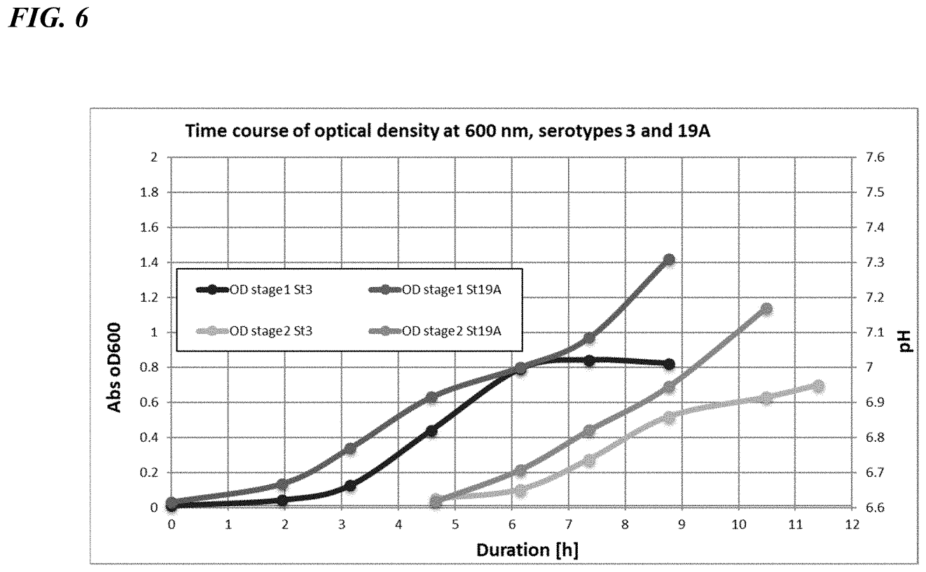

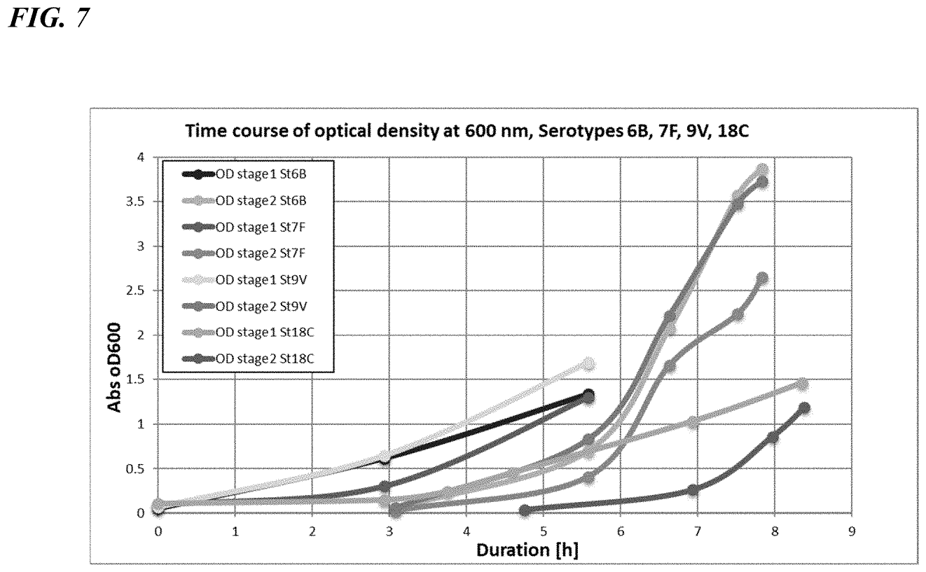

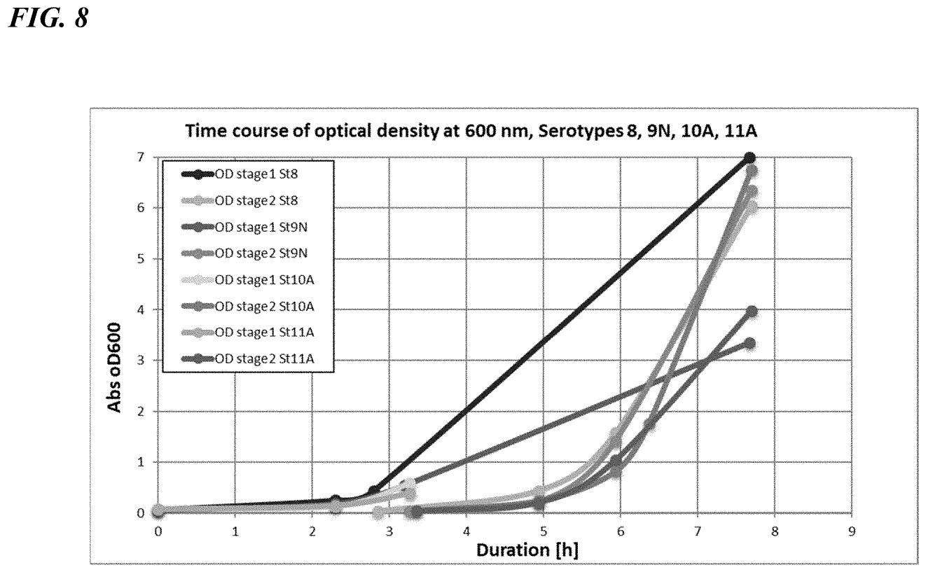

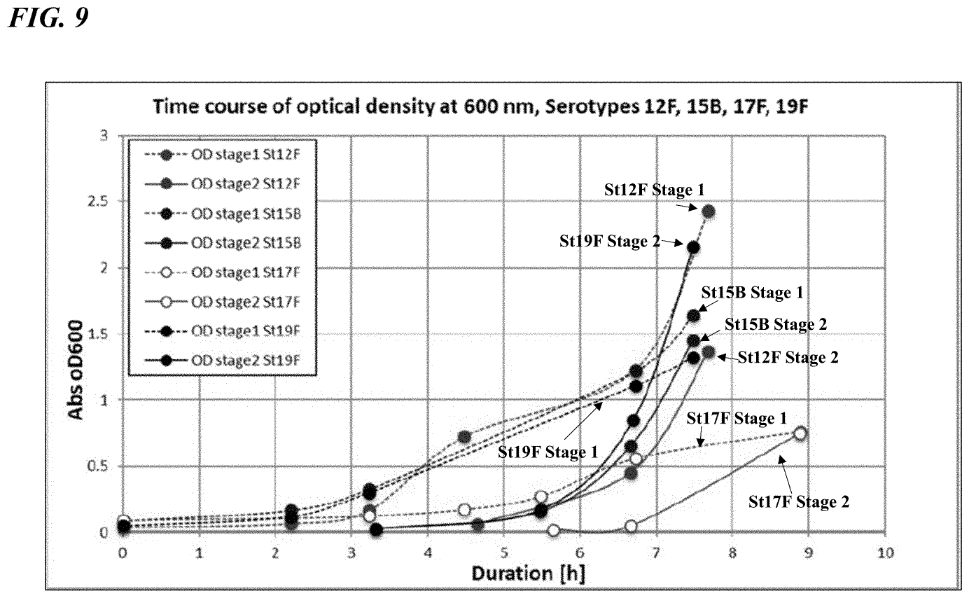

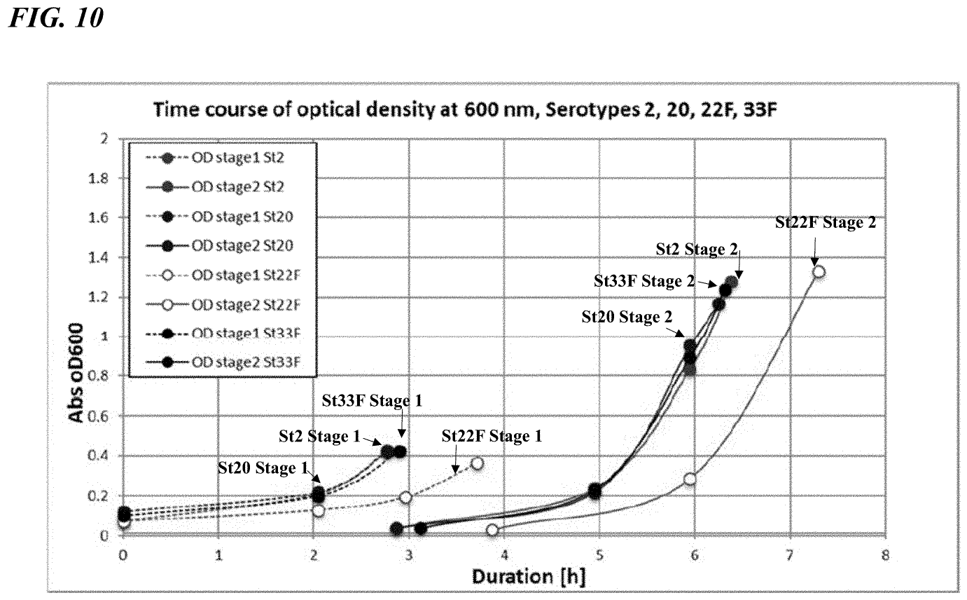

Based on the preliminary experiments, the following conclusions were made and the following procedure for the generation of the parent cell banks was defined:
(a) Media 5 was chosen for generation of the parent cell banks.
(b) Liquid media should be prepared as short as possible prior usage (L-Cysteine containing media should not be older than 2 days). Age of the media may negatively influence the cell growth (FIG. 6 and FIG. 9).
(c) 300 mL shake flasks without baffles with 60 mL media should be used in the first liquid culture stage.
(d) 1000 mL shake flasks without baffles with 180 mL media should be used in the second liquid culture stage.
(e) For inoculation of the first liquid culture stage, 2.4 mL of the cell suspension (up to 200 colonies, depending on the colony size, resuspended in 9 mL of the NaCl 0.9% solution; starting OD600 between 0.02 and 0.1) should be used.
(f) For inoculation of the second liquid culture stage, 18 mL from the first stage at the OD600 above 0.3 (at least one doubling in the first stage is necessary).
(g) Temperature should be 37.0° C.±2.0° C.
(h) Atmosphere should be enriched with CO₂ (5%)
(i) Shaking speed should be 200 rpm at 2 cm shaking diameter (with cooling of the motor). Usage of low heat evolution magnetic stirrer was excluded (see FIG. 8).
(j) The starting pH should be 7.5±0.2 (pH was adjusted during the first experiments with 20% Na₂CO₃ solution)
(k) All measurements of optical density were performed at OD600 with non-inoculated media as a blank (control)
(l) Final optical density prior harvest: 1.0
(m) Addition of the glycerol (30% v/v) to the final concentration of 12% v/v and equilibration for at least 10 min
(n) Filling of 35 vials with 4.5 mL of the culture, initial freezing at −140° C./below −120° C. (further storage at −80° C./below −65° C.)
(o) Positive controls: plating of the cell suspensions used for the inoculation of the first and the second stage either on the SYG or on the TSAII agar with 5% sheep blood and incubation at 37° C. under either anaerobic conditions or in the 5% CO₂ atmosphere.
(p) During purification procedure of each serotype quartet, at least two serotypes were tested for growth behavior in liquid media. See FIG. 5 to FIG. 10.
(q) The level of precipitation of the media was followed by measurement of the OD600 in non-inoculated media in several experiments.

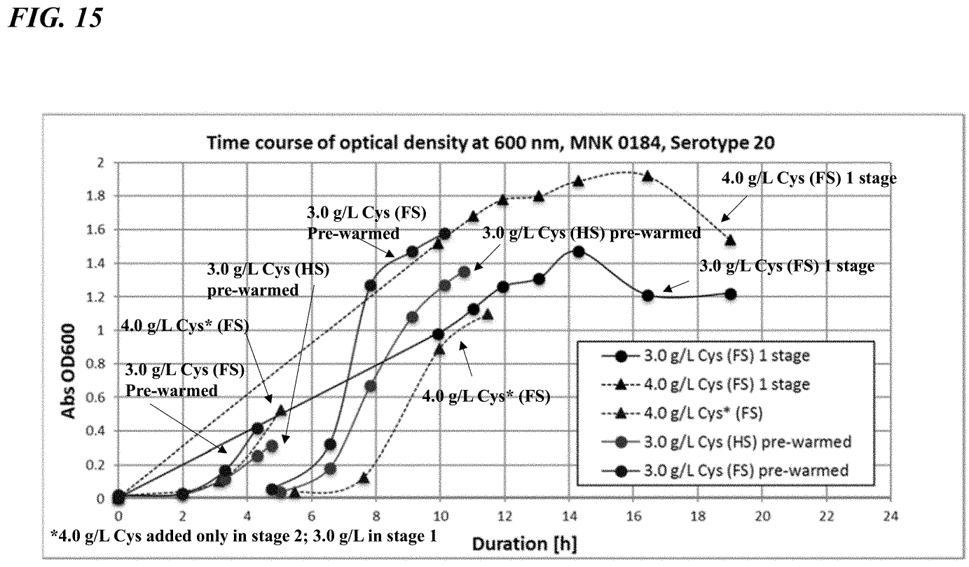

Increasing the concentration of L-cysteine in the medium promotes better growth in flasks. 3.0 g/L of L-cysteine added directly to the medium prior to inoculation was optimal for growth promotion. As shown in FIG. 15, 3.0 g/L and 4.0 g/L of L-cysteine added to the medium for Serotype 20 provided comparable viable cell count and growth performance (time courses of OD600 of serotype 20 in heat (HS) and filter (FS) sterilized media during cultivation experiments in 600 mL scale are shown). However, for most serotypes, 3.0 g/L L-cysteine was found to be the optimal concentration as 4.0 g/L can lead to unwanted precipitation.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of in vitro bacterial cultivation comprising:
a. inoculating an agar medium with catalase-negative bacteria, wherein the agar medium comprises a catalase enzyme and is free of animal-derived materials; and
b. incubating the catalase-negative bacteria on the agar medium under conditions permitting growth of one or more bacterial colonies on the agar medium.

2. The method of claim 1, further comprising:
c. selecting one of the one or more bacterial colonies from the agar medium;
d. inoculating a liquid medium with the selected bacterial colony to produce a liquid bacterial culture;
e. incubating the liquid bacterial culture under growth-permitting conditions; and
f. harvesting cultivated catalase-negative bacteria from the liquid bacterial culture.

3. The method of claim 1, wherein the catalase-negative bacteria is selected from a Streptococcus spp., a Clostriudium spp., an Aerococcus spp., an Enterococcus spp., a Leuconostoc spp., a Pedioccus spp., an Abiotrophia spp., a Granulicatella spp., a Gemella spp., a Rothia mucilaginosa spp., a Lactococcus spp., a Vagococcus spp., a Helcococcus spp., a Globicatella spp., and a Dolosigranulum spp.

4. The method of claim 1, wherein the catalase-negative bacteria is a Shigella spp. selected from S. dysenteriae Type 1 and S. boydii Type 12.

5. (canceled)

6. The method of claim 3, wherein the Streptococcus spp. is a Group A Streptococcus bacteria, a Group C Streptococcus bacteria, or a viridians Streptococcus bacteria.

7. The method of claim 6, wherein the Group A Streptococcus bacteria is S. pyogenes.

8. The method of claim 6, wherein the Group A Streptococcus bacteria is of a serotype selected from M1, M3, M4, M12, M28.

9. The method of claim 3, wherein the Streptococcus spp. is viridians Streptococcus bacteria selected from the mutans group, the salivarius group, the bovis group, the mitis group, and the anginosus group.

10. The method of claim 3, wherein the Streptococcus spp. is S. pneumonia.

11-12. (canceled)

13. The method of claim 10, wherein the S. pneumonia is of a serotype selected from the group consisting of 1, 2, 3, 4, 5, 6A, 6B, 6C, 7C, 7F, 8, 9N, 9V, 10A, 11A, 12F, 14, 15A, 15B, 16F, 17F, 18C, 19A, 19F, 20, 20A, 20B, 21, 22F, 23A, 23B, 23F, 24F, 31, 34, 35B, 33F, and 38.

14. The method of claim 3, wherein the Aerococcus spp. is A. viridians.

15. The method of claim 1, wherein the catalase enzyme is present at a concentration of at least about 500 international units (IU).

16. The method of claim 15, wherein the catalase enzyme is present at a concentration of about 500 IU to about 10000 IU, about 4000 IU to about 6000 IU, about 4500 IU to about 6000 IU, about 5000 IU to about 6000 IU, about 5500 IU to about 6000 IU, about 4000 IU to about 5500 IU, about 4000 IU to about 5000 IU, about 4000 IU to about 4500 IU, about 4500 IU to about 5500 IU, about 4500 IU to about 5000 IU, about 5000 to about 5500 IU, about 4500 IU, about 4600 IU, about 4700 IU, about 4800 IU, about 4900 IU, about 5000 IU, about 5100 IU, about 5200 IU, about 5300 IU, about 5400 IU, or about 5500 IU.

17-19. (canceled)

20. The method of claim 1, wherein the agar medium further comprises a yeast extract, a soy peptone, glucose, one or more salts, and L-cysteine.

21. (canceled)

22. The method of claim 20, wherein the L-cysteine is present at a concentration of at least about 0.5 g/L.

23-26. (canceled)

27. The method of claim 20, wherein the yeast extract is present at a concentration of at least about 5 g/L.

28-29. (canceled)

30. The method of claim 20, wherein the soy peptone is present at a concentration of at least about 5 g/L.

31-38. (canceled)

39. The method of claim 2, wherein the liquid medium comprises substantially the same components as the agar medium.

40-44. (canceled)

45. A cultivated catalase-negative bacteria produced by the method of claim 1, wherein the bacteria demonstrate enhanced polysaccharide production compared to a similar bacteria cultivated using media comprising animal-derived materials.

46. (canceled)

47. A bacterial stock comprising the cultivated catalase-negative bacteria of claim 45.

48. A kit for in vitro bacterial cultivation, comprising:
a. an agar medium that is free of animal-derived materials
b. a catalase enzyme.

49. The kit of claim 48, further comprising a liquid medium comprising substantially the same components as the agar medium.

50. An agarose plate comprising:
a. an agar medium that is free of animal-derived materials; and
b. a catalase enzyme.

51. The agarose plate of claim 50, further comprising catalase-negative bacteria.

52. The bacterial stock of claim 47, further comprising a liquid medium, and, optionally, glycerol, wherein the bacterial stock does not comprise an animal-derived material.

53. The bacterial stock of claim 52, wherein the bacterial stock does not comprise animal-derived heme.

54. The bacterial stock of claim 52, wherein the bacterial stock does not comprise a prion protein, mycoplasma, or viruses.

55. The bacterial stock of claim 52, wherein the bacterial stock demonstrates a decreased amount of cell-wall polysaccharide (CWPS) contamination compared to a bacterial stock comprising a similar bacteria cultivated using media comprising animal-derived materials.
